(19) United States
(12) Patent Application Publication
(10) Pub. No.: US 2026/0347937 A1
(43) Pub. Date: Nov. 5, 2026

(54) Biomarker composition for diagnosing metabolic conditions using antibiotic-resistance mutations in genes or proteins in Akkermansia muciniphila

(71) Applicant: KOREA UNIVERSITY RESEARCH AND BUSINESS FOUNDATION, Seoul (KR)

(72) Inventors: Heenam KIM, Seoul (KR); Yu Min HAN, Seoul (KR)

(21) Appl. No.: 19/434,240

(22) Filed: Dec. 29, 2025

(30) Foreign Application Priority Data
Nov. 14, 2024 (KR) 10-2024-0162504

(51) Int. Cl. C12Q 1/6883; G01N 33/68

(52) U.S. Cl. CPC C12Q 1/6883; G01N 33/6893; C12Q 2600/156; G01N 2333/195; G01N 2800/044

(57) ABSTRACT

According to one embodiment, the present disclosure relates to a composition for diagnosing metabolic disease susceptibility using a gene or promoter variant, or protein variant, a fragment thereof, or a polynucleotide encoding the same in Akkermansia muciniphila, or using the artificial culture characteristics of isolated Akkermansia muciniphila, and a method of providing information for diagnosis.

### BACKGROUND

### 1. Technical Field

The present disclosure relates to a composition for diagnosing metabolic disease susceptibility using an antibiotic resistance mutation in a gene, promoter or protein in Akkermansia muciniphila and a method of providing information for diagnosis.

### 2. Related Art

In recent decades, there has been an increase in various chronic diseases worldwide, a major cause of which has been suggested to be altered gut microbiota in modern human populations. Industrialization and urbanization have been proposed to have cumulatively contributed to the alteration of the microbiota over generations. Among many factors related to industrialization, frequent and excessive exposure to antibiotics since the mass production of penicillin in the early 1940s has been reported to be the most significant contributor to the alteration of the gut microbiome. Specifically, antibiotics have been demonstrated to disrupt the balance of the gut microbiota by altering its community structure, and such disruptions have been shown to lead to improper responses of colonic regulatory T cells, compromised immune tolerance, and increased susceptibility to various disorders, including asthma, allergy, autoimmune disorder, obesity, and vulnerability to opportunistic infections.

Widely used taxonomic-level profiling provides a macroscopic picture of the altered community structure of the gut microbiota. However, assessing additional changes at the individual cellular level is necessary to comprehensively understand the consequences of antibiotics-altered gut microbiota. Such a cellular-level impact may be particularly pronounced when a key microbe such as Akkermansia muciniphila is involved.

Akkermansia muciniphila is a prevalent gut bacterium that resides in the mucus layer of the human colon, and it has the potential to mitigate various chronic diseases, including obesity, type 2 and type 1 diabetes, fatty liver, enteritis, and certain cancers. These potential therapeutic applications have sparked extensive research worldwide

Under this background, during investigation of the effect of penicillin, an antibiotic that was potentially overused extensively during the early stages of the antibiotic era, on Akkermansia muciniphila, the inventors of the present disclosure have discovered that there are many variants of Akkermansia muciniphila that generate and maintain mutation in order to survive. Subsequently, as a result of testing antibiotic-resistant mutants in a mouse model, it was confirmed that most of these mutants lost their ability to promote host health. Therefore, based on these findings, the inventors of the present disclosure have found that the susceptibility of an individual to metabolic disease can be effectively diagnosed by examining whether Akkermansia muciniphila present in the gut of the individual is an antibiotic-resistant mutant or is a wild type, thereby completing the present disclosure.

### SUMMARY

An object of the present disclosure is to provide a composition for diagnosing metabolic disease susceptibility, comprising an agent for detecting a gene or promoter variant, or a protein variant, a fragment thereof, or a polynucleotide encoding the same in Akkermansia muciniphila.

Another object of the present disclosure is to provide a kit for diagnosing metabolic disease susceptibility, comprising the composition.

Still another object of the present disclosure is to provide a method of providing information for diagnosing metabolic disease susceptibility, comprising a step of detecting a gene or promoter variant, or a protein variant, a fragment thereof, or a polynucleotide encoding the same or measuring the expression level thereof in Akkermansia muciniphila in an isolated biological sample.

Yet another object of the present disclosure is to provide a composition for diagnosing gut microbiota imbalance, comprising an agent for detecting a gene or promoter variant, or a protein variant, a fragment thereof, or a polynucleotide encoding the same in Akkermansia muciniphila.

Still yet another object of the present disclosure is to provide a method of providing information for diagnosing gut microbiota imbalance, comprising a step of detecting a gene or promoter variant, or a protein variant, a fragment thereof, or a polynucleotide encoding the same or measuring the expression level thereof in Akkermansia muciniphila in an isolated biological sample.

A further object of the present disclosure is to provide a composition for determining the presence or absence of a mutation in a TEM-type β-lactamase gene promoter or pur gene by isolating Akkermansia muciniphila from a patient and examining the culture characteristics.

Another further object of the present disclosure is to provide a method for determining the presence or absence of a mutation in pur gene, comprising a step of determining whether the growth of Akkermansia muciniphila isolated from a patient is promoted when intermediates of the purine biosynthesis pathway, such as hypoxanthine, are included in a medium during culturing.

Still another further object of the present disclosure is to provide a method for determining the presence or absence of a mutation in a TEM-type β-lactamase gene promoter, comprising a step of determining whether the growth of Akkermansia muciniphila isolated from a patient is promoted when a mucin component such as N-acetyl-D-glucosamine is included in a medium during culturing.

Yet another further object of the present disclosure is to provide a composition for diagnosing the inhibited human-beneficial function of isolated Akkermansia muciniphila, comprising a purine biosynthesis pathway intermediate and a mucin component.

Still yet another further object of the present disclosure is to provide a method of providing information for diagnosing the inhibited human-beneficial function of isolated Akkermansia muciniphila, comprising steps of: determining whether a purine biosynthesis pathway intermediate added during culturing of the isolated Akkermansia muciniphila promotes bacterial growth; determining whether a mucin component added during culturing of the isolated Akkermansia muciniphila promotes bacterial growth; determining whether there is a mutation in a TEM-type β-lactamase gene promoter or pur gene in the isolated Akkermansia muciniphila; determining that there is a mutation in the pur gene, when the purine biosynthesis pathway intermediate added to the medium during culturing of the isolated Akkermansia muciniphila promotes bacterial growth; determining that there is a mutation in the TEM-type β-lactamase gene promoter, when the mucin component added to the medium during culturing of the isolated Akkermansia muciniphila promotes bacterial growth; and determining, based on the presence or absence of the mutation, whether the human-beneficial function of the isolated Akkermansia muciniphila has been inhibited.

According to one embodiment of the present disclosure, there is provided a composition for diagnosing metabolic disease susceptibility, comprising an agent for detecting a gene or promoter variant, or a protein variant, a fragment thereof, or a polynucleotide encoding the same in Akkermansia muciniphila.

According to one embodiment of the present disclosure, the gene or promoter variant may be a TEM-type β-lactamase gene variant.

The protein variant may be a purine biosynthesis protein variant.

According to one embodiment of the present disclosure, the TEM-type β-lactamase gene or promoter variant may include a variant in which nucleotide G in the −10 sequence of the promoter of the gene encoding the TEM-type β-lactamase is replaced with A.

According to one embodiment of the present disclosure, the expression of the TEM-type β-lactamase gene may be significantly increased by the variant in which nucleotide G in the −10 sequence of the promoter of the gene encoding the TEM-type β-lactamase is replaced with A.

According to one embodiment of the present disclosure, the expression of another gene present in the same operon as the TEM-type β-lactamase gene may significantly increase.

According to one embodiment of the present disclosure, the TEM-type β-lactamase protein may comprise the amino acid sequence of SEQ ID NO: 1.

According to one embodiment of the present disclosure, the gene encoding the TEM-type β-lactamase protein may comprise the polynucleotide sequence of SEQ ID NO: 2.

According to one embodiment of the present disclosure, the purine biosynthesis protein variant may be a Pur protein variant.

According to one embodiment of the present disclosure, the Pur protein may be any one or more selected from the group consisting of PurF, PurD, PurN, PurQ, PurS, PurL, PurM, PurE, PurK, PurC, PhrB, and PurH.

According to one embodiment of the present disclosure, the PurF protein variant may comprise a variant in which amino acid Gly at position 53 in the amino acid sequence of the protein is replaced with Asp, or comprise a variant in which 10 nucleotides from the nucleotide at position 2 encoding Tyr610 located at the C-terminus of the amino acid sequence are deleted.

According to one embodiment of the present disclosure, the PurF protein variant may comprise a variant in which GGC nucleotides encoding the amino acid Gly at position 53 in the gene encoding the protein are replaced with GAC nucleotides encoding Asp.

According to one embodiment of the present disclosure, the PurF protein may comprise the amino acid sequence of SEQ ID NO: 3.

According to one embodiment of the present disclosure, the gene encoding the PurF protein y comprise the nucleotide sequence of SEQ ID NO: 4.

According to one embodiment of the present disclosure, the PurQ protein variant may comprise a variant in which amino acid Gly at position 221 in the amino acid sequence of the protein is replaced with Asp.

According to one embodiment of the present disclosure, the PurQ protein may comprise the amino acid sequence of SEQ ID NO: 5.

According to one embodiment of the present disclosure, the gene encoding the PurQ protein may comprise the polynucleotide sequence of SEQ ID NO: 6.

According to one embodiment of the present disclosure, the PurM protein variant may comprise a variant in which amino acid Gln at position 9 in the amino acid sequence of the protein is replaced with Pro, or a variant in which amino acid Gln at position 34 is replaced with a termination codon.

According to one embodiment of the present disclosure, the PurM protein variant may comprise a variant in which CAA nucleotides encoding the amino acid Gln at position 9 in the gene encoding the protein are replaced with CCA nucleotides encoding Pro.

According to one embodiment of the present disclosure, the PurM protein variant may comprise a variant in which CAG nucleotides encoding the amino acid Gln at position 34 in the gene encoding the protein are replaced with the termination codon TAG nucleotides.

According to one embodiment of the present disclosure, the PurM protein may comprise the amino acid sequence of SEQ ID NO: 7.

According to one embodiment of the present disclosure, the gene encoding the PurM protein may comprise the polynucleotide sequence of SEQ ID NO: 8.

According to one embodiment of the present disclosure, the agent may be for detecting a gene or promoter variant, or a protein variant, a fragment thereof, or a polynucleotide encoding the same in Akkermansia muciniphila.

According to one embodiment of the present disclosure, the agent may be selected from the group consisting of a primer, a probe, an antisense oligonucleotide, an antibody, an antigen-binding fragment thereof, or an aptamer, which are capable of specifically amplifying the gene or promoter, or the protein or the polynucleotide encoding the same.

According to one embodiment of the present disclosure, the agent may be any one or more selected from the group consisting of an agent capable of detecting a single nucleotide polymorphism (SNP) in the −10 sequence of the promoter of the nucleotide sequence of SEQ ID NO: 2, an agent capable of detecting a single nucleotide polymorphism (SNP) at nucleotide position 158 in the nucleotide sequence of SEQ ID NO: 4, an agent capable of detecting a single nucleotide polymorphism (SNP) at nucleotide position 662 in the nucleotide sequence of SEQ ID NO: 6, and an agent capable of detecting a single nucleotide polymorphism (SNP) at nucleotide position 26 in the nucleotide sequence of SEQ ID NO: 8.

According to one embodiment of the present disclosure, the agent may be any one or more selected from the group consisting of: a primer or probe capable of amplifying or binding to a polynucleotide consisting of 10 to 100 consecutive nucleotides including a single nucleotide polymorphism (SNP) in the −10 sequence of the promoter of the nucleotide sequence of SEQ ID NO: 2, or a polynucleotide complementary thereto; a primer or probe capable of amplifying or binding to a polynucleotide consisting of 10 to 100 consecutive nucleotides including a single nucleotide polymorphism (SNP) at nucleotide position 158 in the nucleotide sequence of SEQ ID NO: 4, or a polynucleotide complementary thereto; a primer or probe capable of amplifying or binding to a polynucleotide consisting of 10 to 100 consecutive nucleotides including a single nucleotide polymorphism (SNP) at nucleotide position 662 in the nucleotide sequence of SEQ ID NO: 6, or a polynucleotide complementary thereto; and a primer or probe capable of amplifying or binding to a polynucleotide consisting of 10 to 100 consecutive nucleotides including a single nucleotide polymorphism (SNP) at nucleotide position 26 in the nucleotide sequence of SEQ ID NO: 8, or a polynucleotide complementary thereto.

According to one embodiment of the present disclosure, the variant may contain a missense mutation, a frameshift mutation, a nonsense mutation, or a combination of two or more thereof.

According to one embodiment of the present disclosure, the metabolic disease may be obesity, type 2 and type 1 diabetes, hyperlipidemia, hypertension, non-alcoholic and alcoholic fatty liver disease, liver cancer, arteriosclerosis, or enteritis.

According to another embodiment of the present disclosure, there is provided a kit for diagnosing metabolic disease susceptibility comprising the composition.

According to another embodiment of the present disclosure, there is provided a method of providing information for diagnosing metabolic disease susceptibility, comprising a step of detecting a gene or promoter variant, or a protein variant, a fragment thereof, or a polynucleotide encoding the same or measuring the expression level thereof in Akkermansia muciniphila in an isolated biological sample.

According to one embodiment of the present disclosure, the biological sample may be blood, plasma, serum, bone marrow fluid, lymphatic fluid, saliva, lacrimal fluid, mucus, amniotic fluid, or a combination of two or more thereof.

According to one embodiment of the present disclosure, the method of providing information for diagnosing metabolic disease susceptibility may comprise any one or more steps from the group consisting of: a step of identifying a single nucleotide polymorphism (SNP) in the −10 sequence of the promoter of the nucleotide sequence of SEQ ID NO: 2; a step of identifying a single nucleotide polymorphism (SNP) at nucleotide position 158 in the nucleotide sequence of SEQ ID NO: 4; a step of identifying a single nucleotide polymorphism (SNP) at nucleotide position 662 in the nucleotide sequence of SEQ ID NO: 6; and a step of identifying a single nucleotide polymorphism (SNP) t nucleotide position 26 in the nucleotide sequence of SEQ ID NO: 8.

According to one embodiment of the present disclosure, the method may further comprise any one or more steps selected from the group consisting of: a step of diagnosing that the metabolic disease susceptibility is higher when the nucleotide in the −10 sequence of the promoter of the nucleotide sequence of SEQ ID NO: 2 is A than when it is G; diagnosing that the metabolic disease susceptibility is higher when the nucleotide at position 158 in the nucleotide sequence of SEQ ID NO: 4 is A than when it is G; diagnosing that the metabolic disease susceptibility is higher when the nucleotide at position 662 in the nucleotide sequence of SEQ ID NO: 6 is A than when it is G; and diagnosing that the metabolic disease susceptibility s higher when the nucleotide at position 26 in the nucleotide sequence of SEQ ID NO: 8 is C than when it is A.

According to another embodiment of the present disclosure, there is provided a composition for diagnosing gut microbiota imbalance, comprising an agent for detecting a gene or promoter variant, or a protein variant, a fragment thereof, or a polynucleotide encoding the same in Akkermansia muciniphila.

According to another embodiment of the present disclosure, there is provided a kit for diagnosing gut microbiota imbalance comprising the composition for diagnosing gut microbiota imbalance.

According to another embodiment of the present disclosure, there is provided a method of providing information for diagnosing gut microbiota imbalance, comprising a step of detecting a gene or promoter variant, or a protein variant, a fragment thereof, or a polynucleotide encoding the same or measuring the expression level thereof in Akkermansia muciniphila in an isolated biological sample.

According to another embodiment of the present disclosure, there is provided a composition for determining the presence or absence of a mutation in a TEM-type β-lactamase gene promoter or pur gene in an isolated Akkermansia muciniphila, the composition comprising a purine biosynthesis pathway intermediate or a mucin component.

According to one embodiment of the present disclosure, the purine biosynthetic pathway intermediate may be N-hypoxanthine, and the mucin component may be N-acetyl-D-glucosamine, or a combination thereof.

According to another embodiment of the present disclosure, there is provided a method for determining the presence or absence of a mutation in a pur gene in an isolated Akkermansia muciniphila, the method comprising a step of determining whether a purine biosynthesis pathway intermediate promotes bacterial growth.

According to one embodiment of the present disclosure, it may be determined that the mutation is present, when the purine biosynthetic pathway intermediate added to the medium during culturing of the isolated Akkermansia muciniphila promotes bacterial growth.

According to another embodiment of the present disclosure, there is provided a method for determining the presence or absence of a mutation in a TEM-type β-lactamase gene promoter in an isolated Akkermansia muciniphila, the method comprising a step of determining whether a mucin component promotes bacterial growth.

According to one embodiment of the present disclosure, it may be determined that the mutation is present, when the mucin component added to the medium during culturing of the isolated Akkermansia muciniphila promotes bacterial growth.

According to another embodiment of the present disclosure, there is provided a composition for diagnosing the inhibited human-beneficial function of isolated Akkermansia muciniphila, comprising a purine biosynthesis pathway intermediate and a mucin component.

According to another embodiment of the present disclosure, there is provided a method of providing information for diagnosing the inhibited human-beneficial function of isolated Akkermansia muciniphila, comprising steps of: determining whether a purine biosynthesis pathway intermediate and a mucin component promote the growth of Akkermansia muciniphila; determining the presence or absence of a mutation in a TEM-type β-lactamase gene promoter or pur gene through the substance added to a medium during culturing of the isolated Akkermansia muciniphila; determining that the mutation is present, when the purine biosynthesis pathway intermediate or mucin component added to the medium during culturing of the isolated Akkermansia muciniphila promotes bacterial growth; and determining, based on the presence or absence of the mutation, whether the human-beneficial function of the isolated Akkermansia muciniphila has been inhibited.

According to a composition, a kit, and a method of providing information for diagnosing metabolic disease susceptibility using the same according to an embodiment of the present disclosure, a gene or promoter variant or a protein variant in Akkermansia muciniphila, or the growth characteristics of isolated Akkermansia muciniphila can be used to objectively and rapidly detect metabolic disease susceptibility with high accuracy and specificity, and may be effectively used for treatment with a microbiome therapeutic agent after such diagnosis.

### BRIEF DESCRIPTION OF THE DRAWINGS

FIG. 1 shows mutations identified in penicillin-resistant variants of Akkermansia muciniphila. Specifically, FIG. 1a shows a mutation identified in the promoter of a TEM-type β-lactamase gene (INCFGEGE_00238), and shows a single nucleotide substitution of G to A, which enhances the functionality of the −10 sequence. FIG. 1b shows the de novo purine biosynthesis pathway in Akkermansia muciniphila that was disrupted by antibiotic, and the affected enzymes, PurF, PurQSL, and PurM, which are denoted in green letters at their respective enzymatic steps. FIG. 1c relates to details of the mutations identified in pur genes. Here, the mutations in PurF are a single-nucleotide substitution resulting in Gly53Asp and a ten-nucleotide deletion causing consecutive amino acid substitutions starting at Tyr610, the mutation in PurQ is a single-nucleotide substitution leading to Gly221Asp, and the mutations in PurM are single-nucleotide substitutions resulting in Gln9Pro or Gln34Nonsense, leading to premature termination.

FIG. 2 is a diagram showing the chromosomal organization and distribution of purine biosynthesis genes.

FIG. 3 shows that the antibiotic-exposed Akkermansia muciniphila variant exhibited reduced susceptibility to antibiotics. Specifically, FIG. 3a shows the results of qPCR assays conducted on the WT and the strain p*-βL featuring the promoter mutation (mean±S. D., n≥3). ** P<0.01, *** P<0.001 (Student t-test). FIG. 3b shows Etest results of the variants, and shows the results of comparing p*-βL and two strains carrying PurF* (with purF (Tyr610Frameshift)) and PurM* (with purM (Gln9Pro)) compared to the WT (mean±S.D., n≥3). * P<0.05, P<0.01, *** P<0.001 (Student t-test). FIG. 3c shows the results of comparing growth curves of the variants selected after exposure to antibiotics and the WT (mean±S.D., n≥3).

FIGS. 4a to 4f show that antibiotics-exposed Akkermansia muciniphila variants exhibited significantly reduced capacity to mitigate host obesity in the mouse model. Specifically, FIG. 4a relates to the process of establishing the mouse model of obesity, and shows the process of orally administering Akkermansia muciniphila cultures for the last 5 weeks to test the effects of Akkermansia muciniphila strains. FIG. 4b SOWS a differential weight gain among the groups of mice during oral administration, and shows the results of comparing mice administrated with PurF*, PurM*, WT, MucT, and p-βL strains (referred to as m_PurF, m_PurM, m_WT, m_MucT, and m_p*-βL, respectively). FIG. 4c shows the serum levels of LPS in mice. FIG. 4d shows oral glucose tolerance test (OGTT) assays. FIG. 4e shows distinct fat deposition in the liver tissue of mice. FIG. 4f shows the comparison of fat deposition among mouse groups, and specifically, shows comparisons of epididymal, inguinal, mesenteric, retroperitoneal, and perirenal adipose tissues. Photographs of representative mice from each group and their epididymal and mesenteric adipose tissues are shown. * P<0.05, ** P<0.01, *** P<0.001 (Student's t-test).

FIG. 5 shows photographs of mice and their adipose tissues representing each mouse group.

FIG. 6 shows the organization of the operon containing the TEM-type β-lactamase gene (Locus ID: INCFGEGE_00238). Five genes are in this operon.

FIG. 7 shows the global distribution of Akkermansia muciniphila variants. Specifically, FIG. 7a shows the global distribution of strains carrying the promoter mutation of the TEM-type β-lactamase gene. Here, strains carrying the corresponding mutation (G-A) at the SNP position in the −10 sequence of the promoter are denoted with colored boxes in the phylogenetic tree. Also, the sources of these strains are indicated. FIG. 7b shows the global distribution of Akkermansia muciniphila strains carrying mutant Pur proteins truncated by frameshift mutations. Sequences of purF, purQ, purs, purL, and purM from Akkermansia muciniphila strains were acquired from the NCBI database and subjected to analysis to identify mutant proteins. Yellow or green boxes displayed at the ends of the proteins represent amino acid sequences distinct from the wild-type sequences owing to frameshift mutations. The size of the proteins, strain names, and the sources of the strains are shown.

FIG. 8 shows details of mutations identified in pur genes from the NCBI database. Frameshift and nonsense mutations that resulted in the truncation of the encoded proteins are shown.

### DETAILED DESCRIPTION

Hereinafter, the present disclosure will be described in detail. Meanwhile, each description and embodiment disclosed in the present disclosure may also be applied to other descriptions and embodiments. That is, all combinations of various elements disclosed in the present disclosure fall within the scope of the present disclosure. In addition, the scope of the present disclosure may not be considered to be limited by the specific description described below.

Furthermore, those skilled in the art will recognize, or be able to ascertain, using than routine experimentation, numerous equivalents to the specific embodiments described herein. Such equivalents are also intended to be encompassed by the present disclosure.

The present disclosure is based on the novel discovery that metabolic disease susceptibility can be effectively diagnosed using genetic mutations or protein mutations in Akkermansia muciniphila.

Hereinafter, the present disclosure will be described in more detail.

One aspect of the present disclosure provides a composition for diagnosing metabolic disease susceptibility, comprising an agent for detecting a gene or promoter variant, or a protein variant, a fragment thereof, or a polynucleotide encoding the same in an Akkermansia muciniphila strain, or a method for determining the presence or absence of a mutation using the culture characteristics of isolated Akkermansia muciniphila.

As used herein, the term “Akkermansia muciniphila” refers to a prevalent gut bacterium that resides in the mucus layer of the human colon and has the potential to mitigate various chronic diseases, including obesity, type 2 and type 1 diabetes, fatty liver, enteritis, and certain cancers. Akkermansia muciniphila has been reported to be anaerobic in nature and account for approximately 3 to 5% of all gut bacteria.

The term “variant” above means that a mutation has occurred in a gene, gene promoter, or protein present or included in an Akkermansia muciniphila strain. Specifically, the variant may be a gene or promoter variant or protein variant present or included in an Akkermansia muciniphila strain.

The term “protein variant” refers to a product that has a mutation that occurred in the amino acid sequence of wild-type (WT) Akkermansia muciniphila, and thus comprises a sequence partially different from the wild type, or has become truncated, or is abnormal during the production process.

Specifically, the variant may mean one in which at least one amino acid in at least one protein included in Akkermansia muciniphila is substituted, deleted, or added, and thus the protein is rapidly eliminated and rarely exists in cells, or may mean one in which at least one nucleotide in the gene sequence encoding the protein is substituted, deleted, or added.

For example, the gene or promoter variant may be a TEM-type β-lactamase protein variant, the protein variant may be a purine biosynthesis protein variant, and these variants may be present in combination.

In addition, the variant may comprise, but is not limited to, a missense variant, a frameshift variant, a nonsense variant, or a combination of two or more thereof.

The term “TEM-type β-lactamase protein” refers to an enzyme (EC 3.5.2.6) conferring multiple resistance to beta-lactam antibiotics such as penicillin, cephalosporin, cephamycin, monobactam and carbapenem (ertapenem), which is a protein contained in some strains of Akkermansia muciniphila. Beta-lactamase provides antibiotic resistance by breaking the structure of antibiotics. These antibiotics are all known to have a common element in their molecular structure: a four-membered ring known as a β-lactam ring. Through hydrolysis, the protein breaks the β-lactam ring, thereby deactivating the antibacterial properties of the molecule, and the resulting beta-lactamase is typically secreted by Gram-negative bacteria.

The TEM-type β-lactamase gene or promoter variant may comprise a variant in which nucleotide G in the −10 sequence of the promoter of the gene encoding the TEM-type β-lactamase protein is replaced with A.

Specifically, the TEM-type β-lactamase may comprise, consist essentially of, or consist of the amino acid sequence of SEQ ID NO: 1, without being limited thereto.

In addition, the gene encoding the TEM-type β-lactamase protein may comprise, consist essentially of, or consist of the polynucleotide sequence of SEQ ID NO: 2, without being limited thereto.

The TEM-type β-lactamase of the present disclosure may comprise, consist essentially of, or consist of an amino acid sequence or polynucleotide sequence having at least 60% homology or identity to the amino acid sequence of SEQ ID NO: 1 or a gene sequence encoding the same, without being limited thereto. Specifically, the amino acid sequence may comprise an amino acid sequence having at least 60%, 618, 628, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology or identity to SEQ ID NO: 1. In addition, the polynucleotide sequence may comprise a polynucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology or identity to SEQ ID NO: 2.

In addition, it is obvious that any protein having an amino acid sequence having such homology or identity and having deletion, modification, substitution or addition in a portion thereof is also included within the scope of the present disclosure.

That is, although described as “protein or polypeptide having the amino acid sequence set forth in a particular sequence number” or “protein or polypeptide comprising the amino acid sequence set forth in a particular sequence number” in the present disclosure, it is obvious that a protein having an amino acid sequence having deletion, modification, substitution or addition in a portion thereof may also be used in the present disclosure, as long as it has an activity identical or corresponding to that of the polypeptide consisting of the amino acid sequence of the corresponding a sequence number. For example, it is obvious that “polypeptide comprising the amino acid sequence of SEQ ID NO: 1” may also fall within the “polypeptide consisting of the amino acid sequence of SEQ ID NO: 1”, as long as it has an activity identical or corresponding to that of the polypeptide consisting of the amino acid sequence of SEQ ID NO: 1.

As used herein, the term “homology” or “identity” refers to a degree of relatedness between two given amino acid sequences or nucleotide sequences, and may be expressed as a percentage. The term “homology” and the term “identity” may often be used interchangeably with each other.

The sequence homology or identity of conserved polynucleotides or polypeptides may be determined using a standard alignment algorithm, and default gap penalties established by a program to be used may be used together. Substantially, homologous for identical sequences may generally hybridize with each other along at least about 50%, 60%, 70%, 80%, or 90% of the entirety or the full length of the sequences under moderate or highly stringent conditions. In hybridization, polynucleotides containing a degenerate codon instead of a codon are also considered.

Whether any two polynucleotide or polypeptide sequences have homology, similarity, or identity may be determined using known computer algorithms such as the “FASTA” program using, for example, default parameters as in Pearson et al. (1988) Proc. Natl. Acad. Sci. USA 85:2444. Alternatively, this may be determined using the Needleman-Wunsch algorithm (Needleman and Wunsch, 1970, J. Mol. Biol. 48:443-453) as implemented in the Needleman program of the European Molecular Biology Open Software Suite (EMBOSS) package (Rice et al., 2000, Trends Genet. 16:276-277) (version 5.0.0 or later) (GCG program package (including GCG program package (Devereux, J. et al., Nucleic Acids Research 12:387 (1984)), BLASTP, BLASTN, FASTA (Atschul, S. F. et al., J MOLEC BIOL 215:403 (1990); Guide to Huge Computers, Martin J. Bishop, ed., Academic Press, San Diego, 1994, and CARILLO al. et (1988) SIAM J Applied Math 48:1073). For example, the homology, similarity, or identity may be determined using the BLAST of the National Center for Biotechnology Information database, or ClustalW.

The homology, similarity, or identity of polynucleotides or polypeptides may be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program may include: (1) a binary comparison matrix (containing a value of 1 for identities and 0 for non-identities) and the weighted comparison matrix of Gribskov et al. (1986) Nucl. Acids Res. 14:6745, as described by Schwartz and Dayhoff, eds., Atlas of Protein Sequence And Structure, National Biomedical Research Foundation, pp. 353 358 (1979) (EMBOSS version of NCBI NUC4.4); (2) a penalty of 3.0 for each gap and an additional 0.10 penalty for each symbol in each gap (or a gap open penalty of 10 and a gap extension penalty of 0.5); and (3) no penalty for end gaps.

In addition, whether any two polynucleotide or polypeptide sequences have homology, similarity, or identity may be confirmed by comparing the sequences through a Southern hybridization experiment under defined strict conditions, and appropriate hybridization conditions to be defined are within the skill of the art and may be determined by a method well known to those skilled in the art.

The term “purine biosynthesis protein” refers to a protein that biosynthesizes purine nucleotides, and includes a total of 11 Pur proteins, which are encoded by the respective pur genes. The pur genes are known to be similar to their homologs in Escherichia coli. In addition, the pur genes are distributed across the Akkermansia muciniphila chromosome and organized into multiple operons.

Specifically, the purine biosynthesis protein may comprise any one or more proteins selected from the group consisting of amidophosphoribosyltransferase (ATase) protein, phosphoribosylformylglycinamidine synthase protein, and phosphoribosylformylglycinamidine cycloligase protein, without being limited thereto. Specifically, the Pur protein may be any one or more selected from the group consisting of PurF, PurD, PurN, PurQ, PurS, PurL, PurM, PurE, PurK, PurC, PhrB, and PurH.

The term “amidophosphoribosyltransferase (ATase) protein” refers to an enzyme that catalyzes the initial step in the pathway converting 5-phosphoribosyl-1-pyrophosphate (PRPP) into inosine monophosphate (IMP) and may be encoded by purF gene.

The amidophosphoribosyltransferase (ATase) protein may comprise, consist essentially of, or consist of the amino acid sequence of SEQ ID NO: 3, without being limited thereto.

In addition, gene the purF encoding the amidophosphoribosyltransferase (ATase) protein may comprise, consist essentially of, or consist of the polynucleotide sequence of SEQ ID NO: 4, without being limited thereto.

The amidophosphoribosyltransferase (ATase) protein variant may comprise: a variant in which amino acid Gly at position 53 in the amino acid sequence of the protein is replaced with Asp; a variant in which GGC nucleotides encoding the amino acid Gly at position 53 in the gene purF encoding the protein are replaced with GAC nucleotides encoding Asp; or a variant in which 10 nucleotides from the nucleotide at position 2 encoding Tyr610 located at the C-terminus of the amino acid sequence of the protein are deleted.

The “phosphoribosylformylglycinamidine synthase protein” refers to a purine biosynthesis protein that may be encoded by purQSL among 11 pur genes.

Among them, PurQ may comprise, consist essentially of, or consist of the amino acid sequence of SEQ ID NO: 5, without being limited thereto.

In addition, the gene encoding the PurQ protein may comprise, consist essentially of, or consist of the polynucleotide sequence of SEQ ID NO: 6, without being limited thereto.

The PurQ protein variant may comprise a variant in which amino acid Gly at position 221 in the amino acid sequence of the protein is replaced with Asp, or comprise a variant in which GGC nucleotides encoding the amino acid Gly at position 221 in the gene encoding the protein are replaced with GAC nucleotides encoding Asp.

The term “phosphoribosylformylglycinamidine cycloligase protein” refers to a purine biosynthesis protein that may be encoded by purM among 11 pur genes.

The PurM protein may comprise, consist essentially of, or consist of the amino acid sequence of SEQ ID NO: 7, without being limited thereto.

In addition, the gene encoding the PurM protein may comprise, consist essentially of, or consist of the polynucleotide sequence of SEQ ID NO: 8, without being limited thereto.

The PurM protein variant may comprise: a variant in which amino acid Gln at position 9 in the amino acid sequence of the protein is replaced with Pro; a variant in which CAA nucleotides encoding the amino acid Gln at position 9 in the gene encoding the protein are replaced with CCA nucleotides encoding Pro; a variant in which amino acid Gln at position 34 is replaced with a termination codon; or a variant in which CAG nucleotide encoding the amino acid Gln at position 34 in the gene encoding the protein are replaced with the termination codon TAG nucleotides.

The polypeptide or polynucleotide may comprise a polypeptide or polynucleotide fragment comprising the mutation of the purine biosynthesis protein.

The term “fragment” may mean a portion of a polypeptide sequence or a polynucleotide sequence, and specifically may mean a portion comprising a mutated sequence.

The composition may be for detecting a gene encoding a TEM-type β-lactamase protein or a promoter thereof, or a purine biosynthesis protein variant, a fragment thereof, or a polynucleotide encoding the same, or measuring the expression level thereof.

Specifically, the composition may be used to detect a gene encoding a TEM-type β-lactamase protein or a promoter thereof, or a purine biosynthesis protein variant, a fragment thereof, or a polynucleotide encoding the same, or measure the expression level thereof in comparison with the expression level of a control (a gene encoding a TEM-type β-lactamase protein or a promoter thereof, or a wild-type purine biosynthesis protein, a housekeeping protein, etc.), and diagnose metabolic disease susceptibility when the gene encoding TEM-type β-lactamase protein or the promoter thereof, or the purine biosynthesis protein variant is detected or the expression level thereof increases.

Therefore, the composition is capable of diagnosing metabolic disease susceptibility by detecting a gene encoding the TEM-type β-lactamase protein or a promoter thereof, or a purine biosynthesis protein variant, a fragment thereof, or a polynucleotide encoding the same.

As used herein, the term “detection or the measurement of the expression level” means determining whether a specific gene or its promoter, or a protein (peptide) or a fragment thereof, or a gene encoding the protein is expressed or measuring the expression thereof. The expression level of the gene or a promoter thereof, or the protein or a fragment thereof may be the relative or absolute amount of the gene or a promoter thereof, or the protein or a fragment thereof.

Measuring the expression level of the protein or fragment thereof may be measurement of the amount of the protein or fragment thereof.

The expression level of the gene may be the expression level of mRNA encoding the protein. The expression level of of mRNA. mRNA may be the relative or absolute amount Measuring the expression level of the gene may be measurement of the amount of mRNA.

A method for detecting the protein or measuring the expression level thereof may be Western blotting, enzyme-linked immunosorbent assay (ELISA), radioimmunoassay (RIA), radical immunodiffusion, Ouchterlony immunodiffusion, rocket immunoeletrophoresis, immunohistochemical staining, immunoprecipitation assay, complement fixation assay, immunofluorescence, immunochromatography, fluorescence-activated cell sorter (FACS) analysis, or protein chip technology.

A method for detecting the mRNA or gene or measuring the expression level thereof may be reverse transcriptase polymerase chain reaction (RT-PCR), competitive RT-PCR, real-time quantitative RT-PCR, quantitative RT-PCR, RNase protection method, Northern blotting, or DNA chip technology.

As used herein, the term “agent for detecting or measuring the expression level” means a molecule that may be used to detect a specific protein or a gene encoding the same or measure the expression level thereof. Specifically, the agent may comprise an agent capable of detecting and/or amplifying the protein or the gene.

As used herein, the term “agent capable of detecting a specific gene or protein” means an agent capable of specifically binding to and recognizing the specific gene or protein, or detecting and amplifying the specific gene or protein, and the term “agent capable of amplifying a specific gene or protein” means an agent capable of increasing the number of copies of the specific gene or protein by repeating the replication thereof, and for example, may mean a primer capable of specifically amplifying a polynucleotide comprising the gene, or a probe capable of specifically binding to the polynucleotide.

The agent may be an antibody or an antigen-binding fragment thereof, or an aptamer, which specifically binds to the protein variant or fragment thereof. The term “antibody” may be used interchangeably with the term “immunoglobulin”. The antibody may be a polyclonal antibody or a monoclonal antibody. The antibody may be a full-length antibody. The antigen-binding fragment refers to a polypeptide comprising an antigen-binding site. The antigen-binding fragment may be a single-domain antibody, Fab, F(ab′), F(ab′)2, or Fv. The antibody or antigen-binding fragment may be attached to a solid support. The solid support is, for example, a metal chip, a plate, or a surface of a well. The aptamer may be a single-stranded nucleic acid (DNA, RNA, or modified nucleic acid) or a peptide, which specifically binds to the protein or fragment thereof.

The agent may be a nucleic acid comprising a polynucleotide identical or complementary to the gene or promoter variant or identical or complementary to a polynucleotide encoding the protein variant or fragment thereof. The nucleic acid may be a primer, a probe, or an antisense oligonucleotide. The end or inside of the primer, probe, or antisense oligonucleotide may be labeled with a fluorophore, a chemiluminescent substance, or a radioactive isotope.

As used herein, the term “primer” refers to a nucleic acid sequence having a free 3′ hydroxyl group, which may be base-paired with a template complementary to a specific nucleotide sequence and functions as a starting point for copying the template strand. The primer can initiate DNA synthesis in the presence of a reagent for polymerization (i.e., DNA polymerase or reverse transcriptase) and four different nucleoside triphosphates in an appropriate buffer solution at an appropriate temperature. For example, PCR amplification may be performed using sense and antisense primers having a sequence of 7 to 50 nucleotides as specific primers for the mRNA of the gene encoding the biomarker of the present disclosure, and the likelihood of developing a cardiovascular disease in a subject with psoriasis may be predicted by measuring the amount of desired product produced. PCR conditions and the lengths of sense and antisense primers may be appropriately selected according to techniques known in the art. The primers may be 10 to 100, 15 to 100, 10 to 80, 10 to 50, 10 to 30, 10 to 20, 15 to 80, 15 to 50, 15 to 30, 15 to 20, 20 to 100, 20 to 80, 20 to 50, or 20 to 30 nt in length.

As used herein, the term “probe” refers to a nucleic acid fragment such as RNA or DNA that is capable of specifically binding to a target nucleic acid, for example, mRNA. The probe may be labeled so that the presence or absence, content, and expression level of a specific mRNA may be confirmed. The probe may be constructed in the form of an oligonucleotide probe, a single-stranded DNA probe, a double-stranded DNA probe, an RNA probe, etc. For example, hybridization may be performed using a probe having a nucleic acid sequence complementary to the mRNA of a gene encoding the biomarker of the present disclosure, and the expression level of the mRNA may be measured based on the degree of hybridization, thereby predicting the likelihood of developing a cardiovascular disease in a subject with psoriasis. An appropriate probe and hybridization conditions may be appropriately selected according to techniques known in the art. The probe may be 10 to 100, 15 to 100, 10 to 80, 10 to 50, 10 to 30, 10 to 20, 15 to 80, 15 to 50, 15 to 30, 15 to 20, 20 to 100, 20 to 80, 20 to 50, or 20 to 30 nt in length.

In addition, the primer or probe may be chemically synthesized using phosphoramidite solid support synthesis or other well-known methods. Furthermore, the nucleic acid sequence thereof may be modified using various methods known in the art. Examples of such modifications include methylation, capping, substitution with one or more homologs of a natural nucleotide, or modification between nucleotides, such as modification with an uncharged linker (e.g., methyl phosphonate, phosphotriester, phosphoroamidate, carbamate, etc.) or charged linker (e.g., phosphorothioate, phosphorodithioate, etc.). In addition, the primer or probe may also be modified with a label that can directly or indirectly provide a detectable signal. Examples of the label include radioisotopes, fluorescent molecules, or biotin.

In one embodiment, the agent may comprise any one or more selected from the group consisting of an agent capable of detecting a single nucleotide polymorphism (SNP) in the −10 sequence of the promoter of the nucleotide sequence of SEQ ID NO: 2, an agent capable of detecting a single nucleotide polymorphism (SNP) at nucleotide position 158 in the nucleotide sequence of SEQ ID NO: 4, an agent capable of detecting a single nucleotide polymorphism (SNP) at nucleotide position 662 in the nucleotide sequence of SEQ ID NO: 6, and an agent capable of detecting a single nucleotide polymorphism (SNP) at nucleotide position 26 in the nucleotide sequence of SEQ ID NO: 8.

In one embodiment, the agent may comprise any one or more selected from the group consisting of: a primer or probe capable of amplifying or binding to a polynucleotide consisting of 10 to 100 consecutive nucleotides including a single nucleotide polymorphism (SNP) in the −10 sequence of the promoter of the nucleotide sequence of SEQ ID NO: 2, or a polynucleotide complementary thereto; a primer or probe capable of amplifying or binding to a polynucleotide consisting of 10 to 100 consecutive nucleotides including a single nucleotide polymorphism (SNP) at nucleotide position 158 in the nucleotide sequence of SEQ ID NO: 4, or a polynucleotide complementary thereto; a primer or probe capable of amplifying or binding to a polynucleotide consisting of 10 to 100 consecutive nucleotides including a single nucleotide polymorphism (SNP) at nucleotide position 662 in the nucleotide sequence of SEQ ID NO: 6, or a polynucleotide complementary thereto; and a primer or probe capable of amplifying or binding to a polynucleotide consisting of 10 to 100 consecutive nucleotides including a single nucleotide polymorphism (SNP) at nucleotide position 26 in the nucleotide sequence of SEQ ID NO: 8, or a polynucleotide complementary thereto.

As used herein, the term “metabolic disease” is also called metabolic syndrome, and refers to diseases, including heart disease, diabetes, and stroke, in which an individual has three or more of five risk factors (hypertension, hyperglycemia, hypertriglyceridemia, low high-density lipoprotein cholesterol, and central obesity) that increase the risk of health problems. In relation to the metabolic syndrome, hypertension, diabetes, hyperlipidemia, and cardiovascular disease, etc. are increasing explosively. This metabolic syndrome can cause serious complications if left untreated for a long time.

Specifically, the metabolic disease may be, but is not limited to, obesity, hyperlipidemia, hypertension, arteriosclerosis, type 2 and type 1 diabetes, non-alcoholic and alcoholic fatty liver disease, cirrhosis, liver cancer, or enteritis.

As used herein, the term “susceptibility” means a state in which there is a risk of health deterioration due to a lack of resistance to a health risk factor and a vulnerable characteristic. That is, in the present disclosure, metabolic disease susceptibility means a state in which there is a risk of developing metabolic disease due to a lack of resistance to a risk factor of metabolic disease and a vulnerable characteristic.

As used herein, the term “diagnosis” or “diagnosing” means determining whether a specific individual is susceptible to metabolic disease.

As used herein, the term “prediction” or “predicting” means determining whether a specific individual is likely to develop metabolic disease due to high susceptibility to the metabolic disease and, and if so, whether the likelihood of developing the metabolic disease is higher that in many and unspecified persons.

Another aspect of the present disclosure provides a kit for diagnosing metabolic disease susceptibility, comprising the composition for diagnosing metabolic disease susceptibility. The same contents as described above also apply to the kit.

The kit has the effect of diagnosing or predicting metabolic disease susceptibility by detecting the TEM-type β-lactamase promoter or protein variant or purine biosynthesis protein variant, fragment thereof, or polynucleotide encoding the same or measuring the expression level thereof.

The kit may further comprise a sample necessary for predicting metabolic disease susceptibility. The kit may include a solid support, a substrate for immunological detection of an antibody or an antigen-binding fragment thereof, a suitable buffer solution, a chromogenic enzyme, a secondary antibody labeled with a fluorophore, or a chromogenic substrate. The kit may comprise a polymerase, a buffer, a nucleic acid, a coenzyme, a fluorophore, or a combination of two or more thereof, for nucleic acid detection. The polymerase is, for example, Taq polymerase.

Specifically, the kit may be a PCR kit, an RT-PCR kit, a qRT-PCR kit, a DNA chip kit, a Western blot kit, or an ELISA kit.

The PCR kit may be a kit comprising essential elements necessary for performing PCR. In addition to specific polynucleotides, primers or probes for the genes or methylation regions, the PCR kit may comprise a test tube or other appropriate container, a reaction buffer (various pHs and magnesium concentrations), deoxynucleotides (dNTPs), enzymes such as Taq polymerase and reverse transcriptase, DNase, RNAse inhibitors, DEPC water, and sterile water.

The RT-PCR kit may be a kit comprising essential elements required for performing RT-PCR. The RT-PCR kit may comprise, in addition to each primer specific for the genes or methylation region, a test tube or other appropriate container, a reaction buffer (various pHs and magnesium concentrations), deoxynucleotides (dNTPs), dideoxynucleotides (ddNTPs), enzymes such as Taq polymerase and reverse transcriptase, DNase, RNAse inhibitors, DEPC-water, sterile water, etc.

The DNA chip kit may be a kit comprising essential elements required for performing DNA chip assay. The DNA chip kit may include a substrate to which specific polynucleotides, primers or probes for the genes or methylation regions are attached, wherein the substrate may comprise a nucleic acid corresponding to a quantitative control gene or a fragment thereof.

Another aspect of the present disclosure provides a method of providing information for diagnosing metabolic disease susceptibility, comprising a step of detecting or a gene or promoter variant, or a protein variant, a fragment thereof, or a polynucleotide encoding the same or measuring the expression level thereof in Akkermansia muciniphila from a biological sample isolated from a subject.

The same contents as described above also apply to the method.

As used herein, the term “subject” means any organism that is likely to develop metabolic disease. As a specific example, the subject may be a mammal, such as a human, cow, horse, pig, dog, sheep, goat, or cat.

The biological sample refers to a sample obtained from the subject. The biological sample may be, for example, stool, colon and small intestine tissue, blood, plasma, serum, bone marrow fluid, lymphatic fluid, saliva, tears, mucus, amniotic fluid, or a combination of two or more thereof. The biological sample may contain a protein sample or nucleic acid sample obtained from the sample, wherein the nucleic acid sample may comprise DNA, mRNA, or cDNA synthesized from mRNA.

The step of detecting or measuring the expression level may comprise a step of incubating the biological sample with an antibody or an antigen-binding fragment thereof, or an aptamer, which specifically binds to the gene or promoter variant or the protein variant or fragment thereof. The step of measuring may be performed by electrophoresis, immunoblotting, enzyme-linked immunosorbent assay (ELISA), protein chip assay, immunoprecipitation, microarray assay, electron microscopy, or a combination of two or more thereof. The electrophoresis may be SDS-PAGE, isoelectric focusing electrophoresis, two-dimensional electrophoresis, or a combination of two or more thereof.

The step of detecting or measuring the expression level may comprise a step of incubating the biological sample with a polynucleotide identical or complementary to a polynucleotide encoding the gene or promoter variant or the protein variant or fragment thereof. The step of measuring may be performed by Northern blotting or polymerase chain reaction (PCR). The PCR may be real-time PCR or reverse transcription PCR.

The method may further comprise a step of predicting the subject as having a high level of susceptibility to metabolic disease when a gene or promoter variant encoding the TEM-type β-lactamase protein, a purine biosynthesis protein variant, a fragment thereof, or a polynucleotide encoding the protein variant is detected in the isolated biological sample.

The method may further comprise a step of determining that a subject is susceptible to metabolic disease, when a gene or promoter variant encoding the TEM-type β-lactamase protein, or a purine biosynthesis protein variant is detected or expressed.

Alternatively, the method may further comprise a step of determining that the subject is susceptible to metabolic disease, when the expression of the TEM-type β-lactamase gene is dramatically increased by a variant in which nucleotide G the −10 sequence of the promote of the gene encoding the TEM-type β-lactamase is replaced with A, or when the expression of another gene present in the same operon together with the TEM-type β-lactamase gene is also dramatically increased.

In one embodiment, the method may comprise any one or more steps from the group consisting of: a step of identifying a single nucleotide polymorphism (SNP) in the −10 sequence of the promoter of the nucleotide sequence of SEQ ID NO: 2; a step of identifying a single nucleotide polymorphism (SNP) at nucleotide position 158 in the nucleotide sequence of SEQ ID NO: 4; a step of identifying a 662 in the nucleotide sequence of SEQ ID NO: 6; and a step of identifying a single nucleotide polymorphism (SNP) at nucleotide position 26 in the nucleotide sequence of SEQ ID NO: 8.

In one embodiment, the method may further comprise any one or more selected from the group consisting of: a step of diagnosing that the metabolic disease susceptibility is higher when the nucleotide in the −10 sequence of the promoter of the nucleotide sequence of SEQ ID NO: 2 is A than when it is G; a step of diagnosing that the metabolic disease susceptibility is higher when the nucleotide at position 158 in the nucleotide sequence of SEQ ID NO: 4 is A than when it is G; a step of diagnosing that the metabolic disease susceptibility is higher when the nucleotide at position 662 in the nucleotide sequence of SEQ ID NO: 6 is A than when it is G; and a step of diagnosing that the metabolic disease susceptibility is higher when the nucleotide at position 26 in the nucleotide sequence of SEQ ID NO: 8 is C than when it is A.

Another aspect of the present disclosure provides a composition for diagnosing gut microbiota imbalance, comprising an agent for detecting a gene or promoter variant, or a protein variant, a fragment thereof, or a polynucleotide encoding the same in Akkermansia muciniphila.

The terms “Akkermansia muciniphila”, “gene or promoter variant”, “protein variant”, “fragment thereof”, “polynucleotide encoding the same” and “agent for detecting” are as described above.

The term “gut microbiota imbalance” refers to a state in which the number of beneficial gut microbiota is reduced or increased compared to the wild type, resulting in an abnormality in the function of the gut. Specifically, it may refer to an imbalance in the host-beneficial capacities of the gut microorganism Akkermansia muciniphila.

Another aspect of the present disclosure provides a kit comprising a composition for diagnosing gut microbiota imbalance, the composition comprising an agent for detecting a gene or promoter variant, or a protein variant, a fragment thereof, or a polynucleotide encoding the same in Akkermansia muciniphila.

The terms “Akkermansia muciniphila”, “gene or promoter variant”, “protein variant”, “fragment thereof”, “polynucleotide encoding the same”, “kit” and “agent for detecting” are as described above.

Another aspect of the present disclosure provides a composition for determining the presence or absence of a TEM-type β-lactamase gene promoter or pur gene mutation, the composition comprising a purine biosynthesis pathway intermediate or a mucin component, which is added to a medium during culturing of isolated Akkermansia muciniphila.

The terms “Akkermansia muciniphila”, “TEM-type β-lactamase gene promoter”, “pur gene”, “mutation”, “determination”, etc., are as described above.

The purine biosynthesis pathway intermediate refers to an intermediate related to a pathway involved in purine biosynthesis, and specifically may be hypoxanthine. The mucin component may be N-acetyl-D-glucosamine, but various substances may be included in relation thereto, and the present disclosure is not limited to these substances and combinations thereof.

Another aspect of the present disclosure provides a method for determining the presence or absence of a mutation in a pur gene, comprising a step of determining whether a purine biosynthesis pathway intermediate added to a medium during culturing of isolated Akkermansia muciniphila promotes bacterial growth.

Another aspect of the present disclosure provides a method for determining the presence or absence of a mutation in a TEM-type β-lactamase gene promoter, comprising a step of determining whether a mucin composition added to a medium during culturing of isolated Akkermansia muciniphila promotes bacterial growth.

The terms “Akkermansia muciniphila”, “purine biosynthesis pathway intermediate”, “mucin component”, “TEM-type β-lactamase gene promoter”, “pur gene”, “mutation”, “determinant”, etc., are as described above.

It may be determined that the mutation is present, when the purine biosynthesis pathway intermediate or mucin component added to the medium during culturing of the isolated Akkermansia muciniphila promotes bacterial growth.

Another aspect of the present invention provides a composition for diagnosing the inhibited human-beneficial function of isolated Akkermansia muciniphila, comprising a purine biosynthesis pathway intermediate and a mucin component.

Another aspect of the present invention provides a method of providing information for diagnosing the inhibited human-beneficial function of isolated Akkermansia muciniphila, comprising steps of: determining whether a purine biosynthesis pathway intermediate added promotes bacterial growth; determining whether a mucin component promotes bacterial growth; determining whether there is a mutation in a TEM-type β-lactamase gene promoter or pur gene depending on the culture characteristics of isolated Akkermansia muciniphila; determining that there is a mutation in the pur gene, when the purine biosynthesis pathway intermediate or mucin component added to the medium during culturing of the isolated Akkermansia muciniphila promotes bacterial growth; and determining, based on the presence or absence of the mutation, whether the human-beneficial function of the isolated Akkermansia muciniphila has been inhibited.

The terms “Akkermansia muciniphila”, “purine biosynthesis pathway intermediate”, “mucin component”, “TEM-type β-lactamase gene promoter”, “pur gene”, “mutation”, “determination”, etc., are as described above.

Hereinafter, the present disclosure will be described in more detail through examples. These examples are intended to explain the present disclosure in more detail, and the scope of the present disclosure is not limited by these examples.

### Example 1: Obtaining and Culturing of Akkermansia muciniphila Strains

Akkermansia muciniphila strain BAA-835 (MucT) was acquired from ATCC (American Type Culture Collection). An Akkermansia muciniphila strain isolated from the feces of a 24-year-old healthy female residing in Seoul, Korea was designated strain HM1. This study using human-originated Akkermansia muciniphila strains was approved by the Korea University Institutional Review Board (Approval number: KUIRB-2020-0301-01).

The Akkermansia muciniphila strains were cultured in a BHIM medium, which is BHI (brain heart infusion) (Bacto Laboratories, Mt. Pritchard, NSW, Australia) broth supplemented with 0.4% (w/v) mucin from porcine stomach type II (Sigma-Aldrich, St. Louis, MO, USA). Then, the Akkermansia muciniphila cultures were incubated at 37 °C in an anaerobic chamber (Coy Laboratory Products, Grass Lake, Michigan, USA) that maintained anaerobic conditions (N₂ 85%, CO₂ 10%, H₂ 5%). Fully grown cultures were mixed with a 50% volume of sterile, deoxygenated glycerol and stored in a −80 °C freezer.

### Experimental Example 2: Antibiotic Selection of Akkermansia muciniphila Variants

The entire process of this experiment was performed under anaerobic conditions in an anaerobic chamber. Akkermansia muciniphila strain HM1 was grown on BHIM agar plates at 37 °C for 4 days. Then, a single colony was taken and inoculated into 3 ml of BHIM broth, and the culture was incubated for 40 hours at 37 °C. Then, the bacterial cells were centrifuged, washed with BHIM broth, and then adjusted to about 10⁷ CFU/ml. Next, a total of 100 μl of the bacterial suspension was spread on BHIM agar plates containing penicillin G (6 to 12 μg/ml), set at 3 to 4 times the MIC against Akkermansia muciniphila strain HM1. Thereafter, the plates were incubated for 7 days at 37 °C until colonies were formed. Next, these colonies were streaked onto fresh agar plates containing antibiotics to confirm the acquired antibiotic resistance.

### Experimental Example 3: Identification of Mutations Conferring Reduced Antibiotic Susceptibility

To identify mutations in Akkermansia muciniphila variants that confer reduced antibiotic susceptibility, the genomes of the variants that survived antibiotic selection was sequenced using the HiSeq-X platform (Illumina, San Diego, USA). At this time, Data trimming, read mapping to the reference genome, and duplicate read removal were performed using Trimmomatic (v0.38), BWA (v0.7.17), and Sambamba (v0.6.7), respectively. In addition, single nucleotide polymorphism (SNP) analysis was conducted with SAMtools (version 1.9) and BCFtools (version 1.6). In this step, SNPs and short indel candidates with Phred scores above 30 (base call accuracy of 99.9%) were identified using information from the mapped reads.

### Experimental Example 4: Etest Analysis Using Akkermansia muciniphila Strains

The antibiotic susceptibility of Akkermansia muciniphila strains (WT, p*-βL, PurF*, and PurM*) was measured with the Epsilometer test (Etest®) gradient technology (Biomerieux, Marcy-l'Etoile, France).

Specifically, the Etest strips of amoxicillin (AC), ampicillin (AM), benzylpenicillin (penicillin G, PG), and ceftazidime (CZ) were used in the concentration range of 0.016 to 256 μg/ml (AC, AM, and TZ) and 0.002 to 32 μg/ml (PG). Single colonies from the plates were suspended in 2 ml BHI broth until the turbidity reached 4.0 McFarland standard. Next, using sterile cotton swabs, the cell suspensions were spread on BHI agar supplemented plates with mucin (0.4%). After drying the surfaces of the plates for 15 to 20 min, Etest strips were placed on the agar plates. The plates were then incubated at 37 °C for 2 to 6 days until sufficient bacterial growth was observed, and the MIC values were recorded.

### Experimental Example 5: Growth Analysis

A single colony of each Akkermansia muciniphila strain was inoculated into 3 ml BHIM broth, and the culture was incubated at 37 °C in an anaerobic chamber until it reached the stationary phase (OD₆₀₀=about 0.8). The culture was then diluted 1:100 in 40 ml fresh BHIM broth and was incubated at 37 °C. The optical density at 600 nm (OD₆₀₀) was measured every 12 hours for 36 hours using a Nanodrop One cuvette (Thermo Fisher Scientific, Waltham, MA, USA).

### Experimental Example 6: Quantitative Real-Time PCR

Akkermansia muciniphila strains HM1 and p*-βL were cultured in 10 ml BHIM broth at 37 °C in an anaerobic chamber until they reached the stationary phase (OD₆₀₀=0.7 to 1.2). Then, the cultures were inoculated into 80 ml fresh BHIM broth at a ratio of 1:50 and incubated at 37 °C until they reached OD₆₀₀ of 0.4. Next, the cultures were divided, and each of the aliquots was supplemented with penicillin G (final concentration of 1 μg/ml). This is used as a potential inducer for the TEM-type β-lactamase gene at a level lower than the minimum inhibitory concentration (MIC) values for HM1 (2.7 μg/ml) or p*-βL (>32 μg/ml).

Then, cultures with or without the inducer were incubated at 37 °C, and 1 ml samples were taken at 0 and 1 hr and were immediately mixed with 2 ml RNA protect bacteria reagent (Qiagen Sciences Inc., Germantown, MD, USA). The mixtures were centrifuged at 5,000×g for 10 min, and the supernatants were removed. Total RNA was extracted from each cell pellet using the RNeasy Mini Kit (Qiagen Sciences Inc., Germantown, MD, USA) according to the manufacturer's instructions. The total RNA samples extracted were converted into cDNA using M-MLV Reverse Transcriptase (Thermo Fisher Scientific, Waltham, MA, USA) following standard protocols. Quantitative real-time PCR (qRT-PCR) was performed to measure the expression level of the TEM-type β-lactamase gene of Akkermansia muciniphila strains HM1 and p*-βL using Rotor-Gene Q (Qiagen Sciences Inc., Germantown, MD, USA). Then, changes in gene expression were determined using the comparative threshold cycle (CT) method.

Here, primer pairs used for the qRT-PCR were 5′-GGAGCCCTGAATCTGGAACA-3′ and 5′-CACGTGTTGTTGTCGCTCTC-3′ for the TEM-type β-lactamase gene, and 5′-AGGACCTCGTTCTTGCCAAG-3′ and 5′-CAGGGTCTTGATCAGCACGT-3′ for rpoB.

### Experimental Example 7: Establishment of Diet-Induced Obesity (DIO) Mouse Model

A total of 118 four-week-old male C57BL/6N mice were purchased from Koatech (Pyeongtaek, Korea). For the entire 12 weeks of the experiment, the mice were housed in a facility with controlled temperature-humidity and a 12-hr light-dark cycle. Water and diet were provided ad libitum. Next, the mice were fed a normal diet (ND; Altromin International, #1314; with 14% fat, 27% protein, and 59% carbohydrates; 3.34 kcal/g) or a high-fat diet (HFD; Research diets, #D12492; 60% fat, 20% protein, and 20% carbohydrates; 5.24 kcal/g), and body weight was measured once weekly. To minimize environmental influences, all mice were maintained and studied simultaneously. All experiments were approved by the Korea University Institutional Animal Care and Use Committee (KU-IACAC; IACAC-2022-0064).

### Experimental Example 8: Oral Administration of Akkermansia muciniphila Cultures

After 7 weeks of growth (11 weeks-old mice), the mice were randomly divided into 7 groups (n≥10 per group): normal diet only (referred to as ND only); high-fat diet only control (referred to as HFD only); HFD-and MucT-fed (referred to as m_MucT); HFD-and WT-fed (referred to as m_WT); HFD-and p*-βL-fed (referred to as m_p*-βL); HFD-and PurF*-fed (referred to as m_PurF*); and HFD-and PurM*-fed (referred to as m_PurM*).

For 5 weeks, mice were subjected to daily oral administration using an 18-gauge rounded/bulb-tipped gavage needle to inject 200 μl (about 2×10⁸ to 10⁹ cells) of Akkermansia muciniphila culture grown in BHIM broth or just BHIM broth for mice in ND only and HFD only groups.

### Experimental Example 9: Oral Glucose Tolerance Test (OGTT) Assay

After 5 weeks of daily oral administration of Akkermansia muciniphila strains, the mice underwent oral glucose tolerance test (OGTT). The mice were subjected to an 8-hr diet restriction period prior to the test. The baseline glucose concentration was measured before oral administration of glucose (2 g/kg body weight). Following glucose intake, blood was drawn from the tail vein, and the glucose concentration (mg/dl) was measured using an ACCU GUIDE ME glucometer (Roche Diagnostics, Switzerland) at 15-min intervals for 120 min. Measurements were performed in triplicate, and the area under the curve (AUC) was calculated.

### Experimental Example 10: Sampling Blood, Liver, and Adipose Tissues

The mice were fasted overnight for 16 hr and then anesthetized with isoflurane before blood or adipose tissue sampling. Next, blood was collected from the abdominal caval veins using a 1-ml syringe and left for 0.5 to 1 hr in a BD Microtainer Blood Collection tube (SKU: 365, 967, Becton, Dickinson and Company, Franklin Lakes, New Jersey, USA) at room temperature. Then, blood serum was separated from the whole blood by centrifugation at 8000×g, at 4 °C for 10 min. The separated serum was collected, separated into aliquots, and stored at −80 °C.

Next, the epididymal, inguinal, retroperitoneal, perirenal, and mesenteric fat tissues were dissected and weighed. The livers and kidneys were also dissected and weighed. Adipose tissues were placed on plastic-coated paper with a 0.5×0.5-mm grid after washing with sterile 1×PBS (phosphate buffered saline), and then images were taken. After washing with sterile 1×PBS, liver samples were fixed in 10% neutral-buffered formalin solution, and stored at −80 °C for histological analysis.

### Experimental Example 11: Analysis of Serum LPS Levels

The endotoxin lipopolysaccharide (LPS) level was measured using a Pierce Chromogenic Endotoxin Quantification Kit (Thermo Fisher Scientific, Waltham, MA, USA) according to the manufacturer's instructions. First, the serum was diluted (about 20-to 100-fold) in endotoxin-free water. All measurements were performed in duplicate. Marker concentrations were obtained by averaging the two measured concentrations and multiplying the resulting average by the dilution factor.

### Experimental Example 12: Histological Examination

Formalin-fixed liver samples were embedded in paraffin and sliced into 4-μm-thick sections. Then, the slides were stained with Harris hematoxylin and eosin Y solution (H&E; Sigma-Aldrich, Burlington, MA, USA). Frozen liver tissue samples were embedded in an optimal cutting temperature (OCT) compound (Sakura Finetek USA, Inc., Torrance, CA, USA). The OCT-embedded samples were sectioned at a 4-μm thickness and stained with Oil Red O and hematoxylin (Sigma-Aldrich, Burlington, MA, USA). The stained sections were examined and imaged using an optical microscope (BX43F; Olympus Global, Tokyo, Japan).

### Experimental Example 12: Statistical Analysis

For all data, F-test was conducted, followed by t-test. Statistical analysis was performed using IBM SPSS Statistics v27. Statistical significance is indicated by asterisks (*): *p<0.05, ** p<0.01, *** p<0.001, and **** p<0.0001.

### Experimental Example 13: Screening the NCBI Database For Potential Antibiotic-Selected Mutations

To explore the potential global distribution of the promoter mutation of the TEM-type β-lactamase gene identified in this experiment, BLASTN searches were conducted against the NCBI database (https://blast.ncbi.nlm.nih.gov/Blast.cgi) using a DNA sequence (200 bp) covering the promoter region and the 5′-end portion of the TEM-type β-lactamase gene. As a result, sequences from A. muciniphila strains carrying the A allele at the SNP position in the −10 sequence within the promoter were identified. Associated TEM-type β-lactamase homologs were retrieved from the NCBI, and they were used to construct a phylogenetic tree initially by aligning the AA sequences using CLUSTALX 2.1 with the “Do Complete Alignment” option and default settings, then by uploading the final file to the ITOL website (https://itol.embl.de/) for tree construction and annotation. To identify strains with mutated Pur enzymes, segments of about 50 amino acid sequences from both the N-terminal and middle portions of PurF, PurQ, PurS, PurL, and PurM were used for BLASTP searches in the NCBI database. Proteins significantly shorter than their wild-type counterparts were retrieved, and their corresponding genes were inspected to identify frameshift or nonsense mutations.

### Example 1. Identification of Akkermansia muciniphila Variants after Exposure to Antibiotics

To investigate the impact of antibiotics on Akkermansia muciniphila, a Akkermansia muciniphila strain isolated from a healthy individual (referred to as strain HM1 or the wild type (WT) in this experiment) was exposed to a lethal dose of penicillin G (6 to 12 μg/ml). Then, the genomes of the WT strain and the Akkermansia muciniphila variants selected after exposure to penicillin G were sequenced to identify genetic changes in the variants.

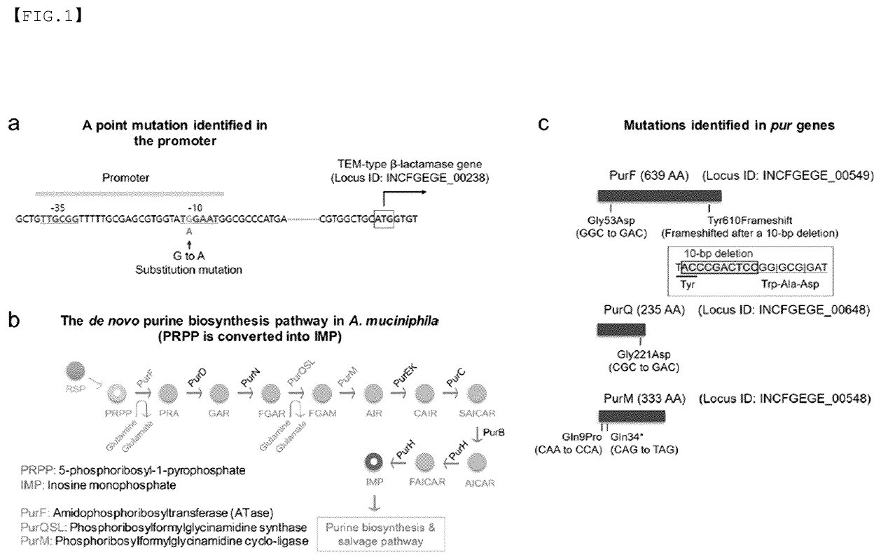

As a result, as can be seen in FIG. 1a, one among the mutations could be identified in the region upstream of a TEM-type β-lactamase gene (INCFGEGE_00238), which is the promoter (FIG. 1a). This mutation involves a single-nucleotide substitution, in which nucleotide G is replaced with A. This was within the −10 sequence of the promoter, with a high potential to enhance promoter activity by increasing the A·T content of the −10 sequence (FIG. 1a).

Next, as can be seen in FIG. 1b, mutations were identified in several pur genes, namely purF, purQ and purM (FIG. 1b). These genes encode enzymes in the de novo purine biosynthesis pathway, a major pathway in nucleotide metabolism (FIG. 1b). purF encodes amidophosphoribosyltransferase (ATase), which catalyzes the initial step in the pathway converting 5-phosphoribosyl-1-pyrophosphate (PRPP) into inosine monophosphate (IMP). purQ, along with purs and purL, encodes phosphoribosylformylglycinamidine synthase, and purM encodes phosphoribosylformylglycinamidine cycloligase.

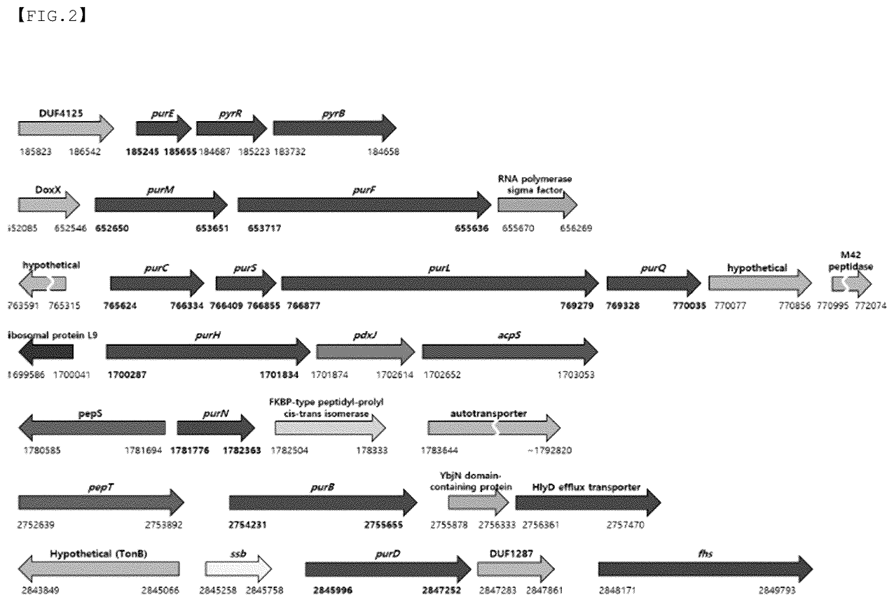

Eleven pur genes encode the de novo purine biosynthesis pathway, similar to their homologs in Escherichia coli (FIGS. 1b and 2). The pur genes were distributed across the Akkermansia muciniphila chromosome and organized into multiple operons (FIG. 2).

Two mutations were found in purF: a single-nucleotide substitution resulting in Gly53Asp ten-nucleotide deletion that caused a frameshift, resulting in the replacement of C-terminal 30 amino acids, starting at Tyr610 (FIG. 1c).

The mutation found in purQ was a single-nucleotide substitution resulting in Gly221Asp, and the two mutations identified in purM were single-nucleotide substitutions, resulting in Gln9Pro and premature termination at Gln34 in the enzyme (FIG. 1c). This premature termination occurred due to the conversion of the Gln-encoding CAG into a nonsense codon T (U) AG (FIG. 1c). Unlike frameshift or nonsense mutations, the impact of single amino acid substitutions needs to be further examined. However, considering that the frameshift mutation in purF and the nonsense mutation in purM had a serious effect on the respective enzymes, it is likely that these missense mutations resulted in substantial disruptions in the respective enzymes (FIG. 1c).

To assess whether the predicted enhancement of promoter activity occurred due to the G to A single-nucleotide substitution, quantitative PCR (qPCR) assays were conducted using a variant strain carrying this mutation (p*-BL) and the WT strain.

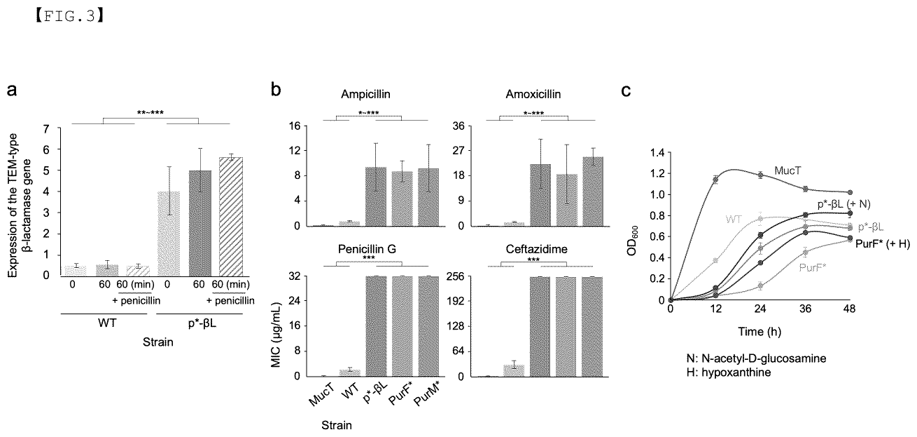

As a result, as can be seen in FIG. 3a, it was confirmed that the mutation significantly increased the basal-level expression of the TEM-type β-lactamase gene, as measured at two different time points (0 and 1 hr) during incubation of the bacterial cultures within the log phase (FIG. 3a). However, half of the cultures exposed to low levels of penicillin G (final concentration of 1 μg/ml) did not exhibit a significant induction in the gene regardless of the presence of the mutation (FIG. 3a).

In addition, consistent with the qPCR results, Etest results with strain p*-βL exhibited significantly elevated minimum inhibitory concentrations (MICs) for penicillin G, and additional β-lactam antibiotics (amoxicillin, ampicillin, and ceftazidime), compared to the WT (FIG. 3b).

Next, two strains carrying a mutation in two pur genes, that is, PurF* (variant carrying purF (Tyr610Frameshift)) and PurM* (variant carrying purM (Gln9Pro)), were assessed for their susceptibility to these antibiotics. As a result, as can be seen in FIG. 3b, it could be confirmed that the Etest results showed significantly reduced susceptibility of the two variants to antibiotics compared to the WT (FIG. 3b).

Next, the growth of the mutants was compared to that of the WT strain. As a result, as can be seen in FIG. 3c, it could be confirmed that the growth of mutant strain p*-βL was nearly identical to that of the WT strain, while PurF* and PurM* variants, particularly the former variant, exhibited significantly slower growth compared to the WT strain. These results suggest a severely affected physiology of these purine variants (FIG. 3c).

### Example 2: Confirmation of the Reduced Ability of Akkermansia muciniphila Variants to Mitigate Obesity

To assess the potential impact of the antibiotics-selected mutations, the host-beneficial capacity of the Akkermansia muciniphila variants was compared with that of the wild-type strain in mice.

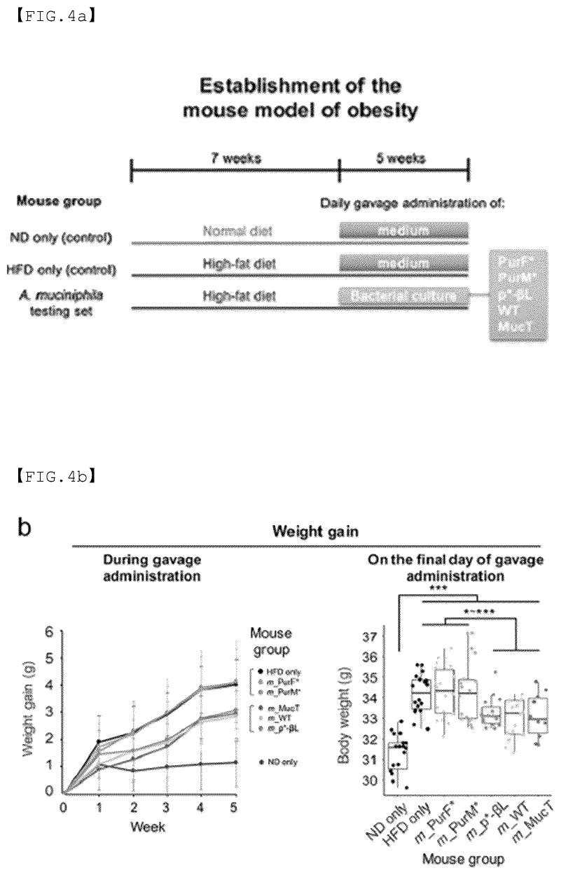

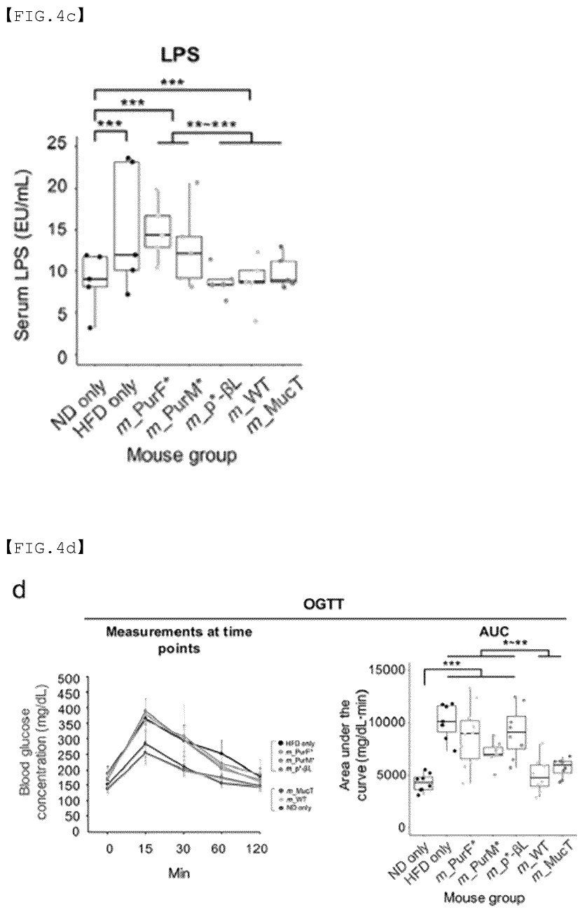

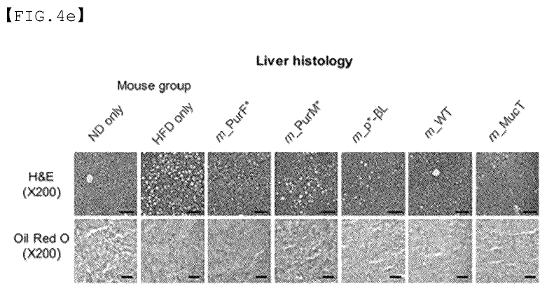

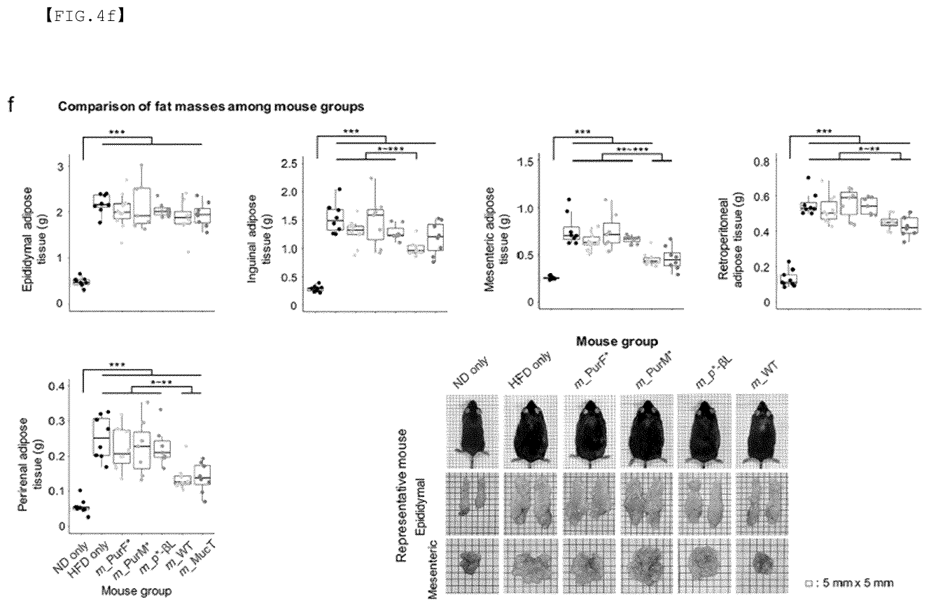

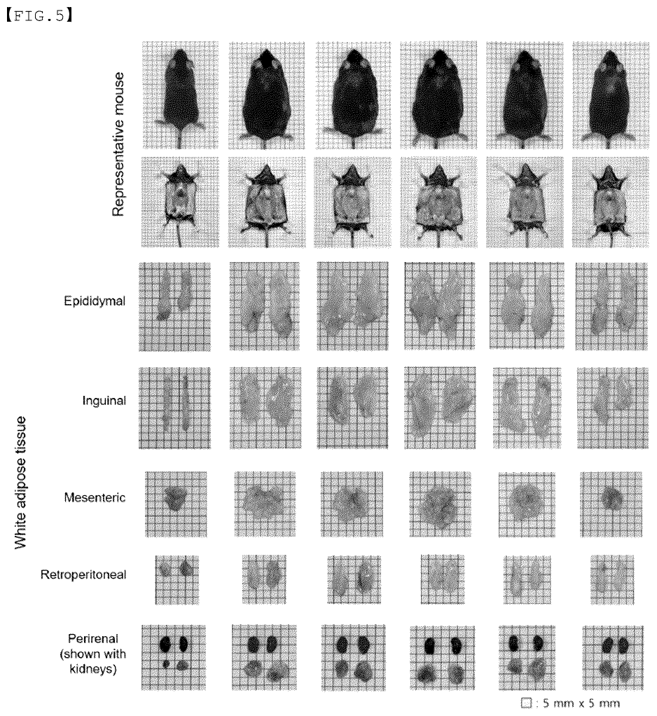

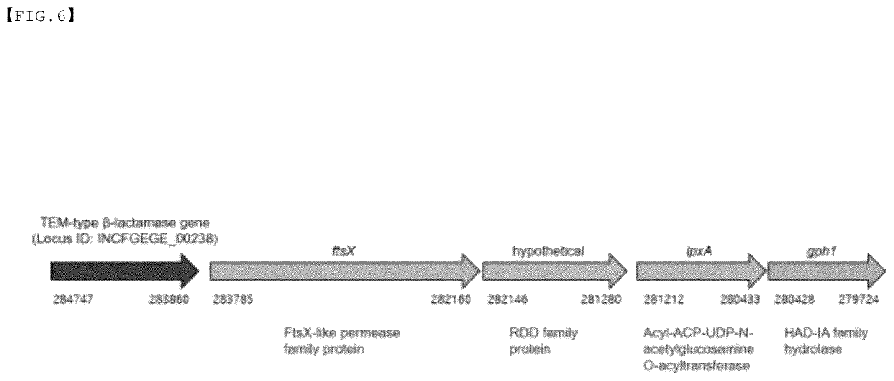

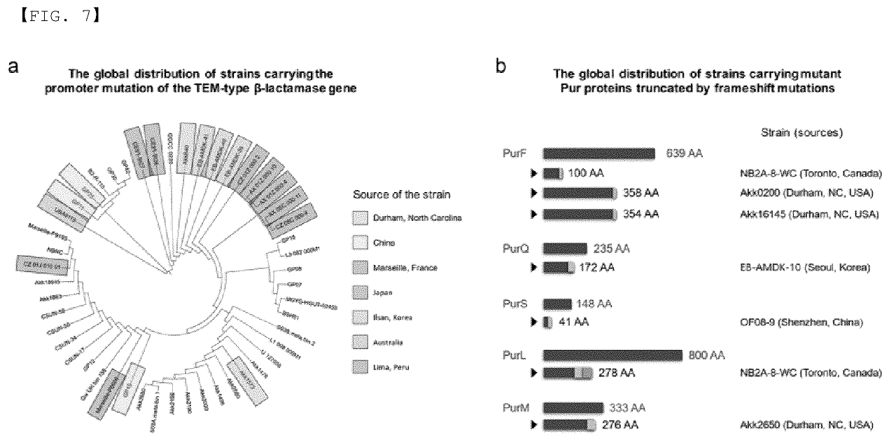

To this end, a mouse model of obesity was developed by subjecting mice to a high-fat diet for 12 weeks (FIGS. 4a to 4f). During the last 5 weeks of this period, these mice were subjected to daily oral administration of either an Akkermansia muciniphila culture or growth medium as a control (FIG. 4a). The tested strains included the type strain MucT (ATCC BAA-835), whose host-beneficial activity has been demonstrated, the WT strain, and the following variants: p*-βL (strain with the promoter mutation), PurF* (strain carrying purF (Tyr610Frameshift)), and PurM* (strain carrying purM (Gln9Pro)).

As a result, as can be seen in FIGS. 4b, 4c and 5, it could be confirmed that a mouse group fed a high-fat diet (HFD) without oral administration of Akkermansia muciniphila (referred to as “HFD only” mice) exhibited a significantly higher increase in body weight compared to a mouse group fed a normal diet (ND) (referred to as “ND only” mice) (FIGS. 4b, 4c and 5).

However, it could be confirmed that, even among the mouse groups fed a high-fat diet, the mice orally administered with the MucT or WT strain (referred to as m_MucT or m_WT) exhibited significantly lower weight gain, consistent with the findings of previous studies (FIGS. 4b and 4c). In particular, it was confirmed that mice fed a high-fat-diet and administered orally with mutant strains PurF* (referred to as m_PurF*) or PurM* (referred to as m_PurM*), and p*-βL mice (referred to as m_p*-βL), failed to exhibit the weight reduction exhibited by the m_MucT or m_WT mice (FIGS. 4b and 5). This suggests that the variants with pur mutations have a reduced ability to mitigate host obesity. Furthermore, the weight gain in the mice strongly correlated with elevated serum lipopolysaccharide (LPS) levels as described below, suggesting the onset of endotoxemia in these obese mice (FIG. 4c).

Specifically, it was confirmed that, in oral glucose tolerance tests (OGTTs), m_PurF* and m_PurM* mice exhibited significantly compromised glucose tolerance compared to the m_MucT and m_WT mice (FIG. 4d). In particular, it was confirmed that m_p*-βL mice also showed compromised glucose tolerance that was similar to m_PurF* and m_PurM* mice (FIG. 4d).

In addition, it could be confirmed that the fat deposition displayed in the liver histology of obese mice resembled that observed in hepatocellular steatosis, a hallmark of nonalcoholic fatty liver disease (NAFLD) (FIG. 4e). Furthermore, it could be confirmed that fat deposition throughout the body was evident in obese mice (FIGS. 4f and 5). In particular, it could be confirmed that these mice displayed significantly larger adipose tissues throughout the body than m_WT or m_MucT mice, and the epididymal adipose tissues were highly similar among all high-fat-diet-fed mice (FIGS. 4f and 5). Among them, m_p*-βL mice displayed distinct characteristics compared to m_WT or m_MucT mice, showing similar patterns to m_PurF* and m_PurM* mice (FIG. 4f), despite the absence of significant overall weight gain (FIG. 4b).

These results indicate that Akkermansia muciniphila variants selected after exposure to antibiotics exhibited a reduced host-beneficial capacity, and in particular, purine metabolism-defective variants, PurF* and PurM* variants, show an enhanced reduction compared to the promoter variant p*-βL.

These results suggest that PurF* and PurM* variants and the promoter variant p*-βL may be used as biomarkers for diagnosing metabolic disease susceptibility or as biomarkers for diagnosing gut microbiota imbalance.

### Example 3: Assessment of Global Prevalence of Antibiotics-Selected Akkermansia muciniphila Variants

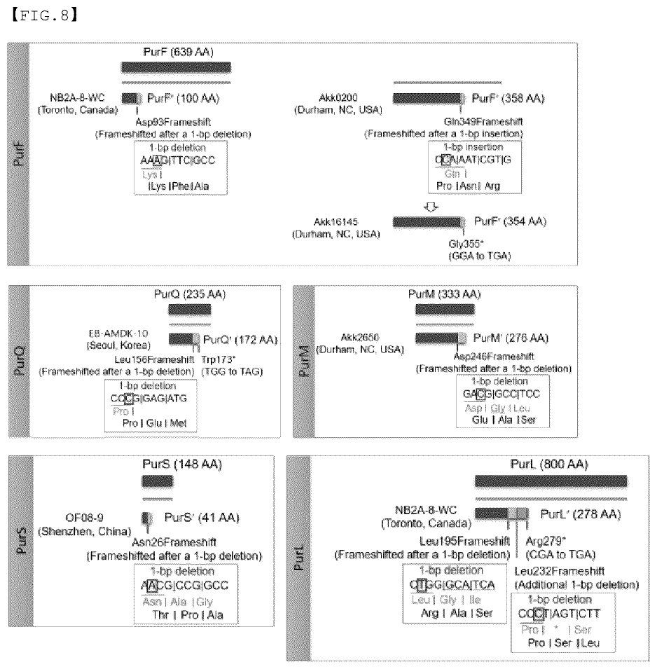

To assess the worldwide prevalence of the single nucleotide polymorphism (SNP) resulting from the G to A substitution in the promoter of the TEM-type β-lactamase gene in Akkermansia muciniphila strains, the present inventors searched for the corresponding sequence in the National Center for Biotechnology Information (NCBI) database. As confirmed in the Examples described above, two possible nucleotide variations in the SNP were G and A. While the G allele, corresponding to the wild type, was more abundant, the A allele, corresponding to the mutation, was also prevalent globally among Akkermansia muciniphila strains across diverse regions, including the USA, France, Australia, Peru, Japan, China, and Korea (FIG. 7a and Table 1 below).

TABLE 1

| | | | Geographical | | |
|---|---|---|---|---|---|
| [symbol] | [symbol] | [symbol] | location | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | |
| [symbol] | [symbol] | [symbol] | [symbol] | [symbol] | [symbol] |

[symbol]

indicates data missing or illegible when filed

As can be seen in FIG. 7a, it was confirmed that some closely related strains, as observed in the phylogenetic tree constructed using TEM-type β-lactamase homologs, shared the A allele, indicating that the mutation was inherited within these lineages (FIG. 7a). Conversely, it was confirmed that some strains did not display the A allele shared by related strains, suggesting that their mutations have recently emerged (FIG. 7a). These findings suggest that the antibiotic selection observed in vitro has global in vivo prevalence and that the mutations have been inherited within Akkermansia muciniphila lineages in the human gut.

Subsequently, the present inventors identified potential signatures of antibiotics-selected pur gene mutations from the DNA sequences of Akkermansia muciniphila strains available in the NCBI database. First, identifying the promoter mutation was straightforward because of its specific base change at the designated location (the −10 sequence) (FIG. 1a). However, pinpointing mutations in the pur gene was more intricate due to their diverse nature. This diversity spans multiple target genes, such as purF, purQ, purM, and potentially other pur genes, and encompasses various modes of disruption, including missense, nonsense, and frameshift mutations (FIGS. 1b and 1c). Therefore, distinguishing antibiotics-selected mutations from natural variations based solely on nucleotide sequences is challenging. Therefore, the present inventors focused on identifying frameshift and nonsense mutations in PurF, PurQSL, and PurM, which resulted in evident disruptions of the enzymes.

As a result, as can be seen in FIG. 7b, it was confirmed that three strains sharing the same frameshift mutation could be found, and two of them were closely related (FIG. 7b). Specifically, it was confirmed that strain Akk16145 shared the same frameshift mutation found in purF as strain Akk0200, leading to the insertion of different amino acids in PurF, starting at Gln349 (FIG. 8). Strain Akk16145 carried its own nonsense mutation downstream, resulting in the truncation from Gly355. This suggests that strain Akk16145 is a descendant of strain Akk0200.

In fact, the two strains originated from the same geographical location in Durham, North Carolina, USA (FIG. 7b and Table 2 below).

TABLE 2

| Strain | | | | | |
|---|---|---|---|---|---|
| (sample name) | ACCESSION | DBSOURCE | Gene | BioSample | BioProject |
| NB2A-8-WC | UQT45456.1 | CP097283.1 | purF | SAMN28102047 | PRJNA835435 |
| Akk0200 | WP_257228631.1 | NZ_CP072050.1 | purF | SAMN18350234 | PRJNA715455 |
| Akk16145 | WP_257228177.1 | NZ_CP072031.1 | purF | SAMN18350254 | PRJNA715455 |
| EB-AMDK-10 | WP_260735522.1 | NZ_CP025825.1 | purQ | SAMN08329147 | PRJNA429085 |
| OF08-9 | WP_270589558.1 | NZ_JAQEVJ010000016.1 | purS | SAMN31809641 | PRJNA903559 |
| NB2A-8-WC | UQT45595.1 | CP097283.1 | purL | SAMN28102047 | PRJNA835435 |
| Akk2650 | WP_257227633.1 | NZ_JAGFOC010000002.1 | purM | SAMN18353368 | PRJNA715455 |

| Strain | Geographical | | | |
|---|---|---|---|---|
| (sample name) | location | Source | w/frameshift | URL |
| NB2A-8-WC | Toronto, Canada | Human feces | Y | https://www.ncbi.nlm.nih.gov/ipg/UQT45456.1 |
| Akk0200 | Durham, North | Human feces | Y | https://www.ncbi.nlm.nih.gov/ipg/WP_257228631.1 |
| | Carolina, USA | | | |
| Akk16145 | Durham, North | Human feces | Y | https://www.ncbi.nlm.nih.gov/ipg/WP_257228177.1 |
| | Carolina, USA | | | |
| EB-AMDK-10 | Seoul, Korea | Human feces | Y | https://www.ncbi.nlm.nih.gov/ipg/WP_260735522.1 |
| OF08-9 | Shenzhen, China | Human feces | Y | https://www.ncbi.nlm.nih.gov/ipg/WP_270589558.1 |
| NB2A-8-WC | Toronto, Canada | Human feces | Y | https://www.ncbi.nlm.nih.gov/ipg/UQT45595.1 |
| Akk2650 | Durham, North | Human feces | Y | https://www.ncbi.nlm.nih.gov/ipg/WP_257227633.1 |
| | Carolina, USA | | | |

Next, the present inventors identified a strain (NB2A-8-WC) carrying a mutant purL in which three consecutive mutations were observed: two frameshift mutations and a nonsense mutation (FIGS. 7b and 8). It is likely that these mutations arose consecutively in this gene over generations. Numerous mutant pur genes could be identified from such limited database resources, and even though the present inventors examined only a few pur genes and did not consider potentially abundant missense mutations, it could be seen that Akkermansia muciniphila strains carrying mutant pur genes are likely to be globally prevalent.

In summary, although there is no direct evidence that the promoter and pur gene mutations identified in the Akkermansia muciniphila database resulted from antibiotic selection, many of them likely enhance bacterial survival to antibiotics. Considering that humans have been extensively exposed to antibiotics over the past few decades, this likelihood is quite high. These mutations do not benefit bacteria beyond conferring survival to antibiotics, and hinder their growth as demonstrated in this study (FIG. 3c). Importantly, these mutations seem to have been inherited within Akkermansia muciniphila lineages (FIGS. 7 and 7b). This aligns with the understanding that mutations persist within the bacterial community owing to their slow reversibility. This phenomenon is attributed to compensatory evolution, cost-free mutations, and genetic co-selection, even if the mutants themselves incur a fitness cost in the form of reduced growth rate.

Consequently, it is likely that antibiotics-selected variants of Akkermansia muciniphila persist in human microbiota, potentially influencing human health. This further implies the possibility that these Akkermansia muciniphila variants have contributed to global epidemics of chronic conditions, offering insights into the missing links with altered microbiota in the modern human population.

While the present disclosure has been described, it will be understood by those skilled in the art to which the present disclosure pertains that the present disclosure may be embodied in other specific forms without departing from the technical spirit or essential characteristics of the present disclosure. Therefore, the embodiments described above are considered to be illustrative in all respects and not restrictive. The scope of the present disclosure is defined by the appended claims rather than the detailed description, and it should be understood that all modifications or variations conceived from the meanings and scope of the appended claims and equivalents thereto are included in the scope of the present disclosure.

What is claimed is:

1. A composition for diagnosing metabolic disease susceptibility, comprising an agent for detecting a gene or promoter variant, or a protein variant, a fragment thereof, or a polynucleotide encoding the same in an Akkermansia muciniphila.

2. The composition of claim 1, wherein the gene or promoter variant is a TEM-type β-lactamase variant.

3. The composition of claim 1, wherein the protein variant is a purine biosynthesis protein variant.

4. The composition of claim 2, wherein the TEM-type β-lactamase variant comprises a variant in which nucleotide G in a −10 sequence of a promoter of a gene encoding the TEM-type β-lactamase is replaced with A.

5. The composition of claim 4, wherein the TEM-type β-lactamase comprises the amino acid sequence of SEQ ID NO: 1.

6. The composition of claim 4, wherein the gene encoding the TEM-type β-lactamase comprises the polynucleotide sequence of SEQ ID NO: 2.

7. The composition of claim 3, wherein the purine biosynthesis protein variant is a Pur protein variant.

8. The composition of claim 7, wherein the Pur protein is any one or more selected from the group consisting of PurF, PurD, PurN, PurQ, PurS, PurL, PurM, PurE, PurK, PurC, PhrB, and PurH.

9. The composition of claim 8, wherein the PurF protein variant comprises a variant in which amino acid Gly at position 53 in the amino acid sequence of the protein is replaced with Asp, or a variant in which 10 nucleotides from the nucleotide at position 2 encoding Tyr610 located at the C-terminus of the amino acid sequence are deleted.

10. The composition of claim 8, wherein the PurF protein comprises the amino acid sequence of SEQ ID NO: 3.

11. The composition of claim 8, wherein the PurQ protein variant comprises a variant in which amino acid Gly at position 221 in the amino acid sequence of the protein is replaced with Asp.

12. The composition of claim 8, wherein the PurQ protein comprises the amino acid sequence of SEQ ID NO: 5.

13. The composition of claim 8, wherein the PurM protein variant comprises a variant in which amino acid Gln at position 9 in the amino acid sequence of the protein is replaced with Pro, or a variant in which amino acid Gln at position 34 is replaced with a termination codon.

14. The composition of claim 8, wherein the PurM protein variant comprises a variant in which CAA nucleotides encoding the amino acid Gln at position 9 in the gene encoding the protein are replaced with CCA nucleotides encoding Pro.

15. The composition of claim 8, wherein the PurM protein comprises the amino acid sequence of SEQ ID NO: 7.

16. The composition of claim 1, wherein the agent is any one or more selected from the group consisting of an agent capable of detecting a single nucleotide polymorphism (SNP) in the −10 sequence of the promoter of the nucleotide sequence of SEQ ID NO: 2, an agent capable of detecting a 158 in the nucleotide sequence of SEQ ID NO: 4, an agent capable of detecting a single nucleotide polymorphism (SNP) at nucleotide position 662 in the nucleotide sequence of SEQ ID NO: 6, and an agent capable of detecting a single nucleotide polymorphism (SNP) at nucleotide position 26 in the nucleotide sequence of SEQ ID NO: 8.

17. The composition of claim 1, wherein the agent is any one or more selected from the group consisting of:
a primer or probe capable of amplifying or binding to a polynucleotide consisting of 10 to 100 consecutive nucleotides including a single nucleotide polymorphism (SNP) in the −10 sequence of the promoter of the nucleotide sequence of SEQ ID NO: 2, or a polynucleotide complementary thereto;
a primer or probe capable of amplifying or binding to a polynucleotide consisting of 10 to 100 consecutive nucleotides including a single nucleotide polymorphism (SNP) at nucleotide position 158 in the nucleotide sequence of SEQ ID NO: 4, or a polynucleotide complementary thereto;
a primer or probe capable of amplifying or binding to a polynucleotide consisting of 10 to 100 consecutive nucleotides including a single nucleotide polymorphism (SNP) at nucleotide position 662 in the nucleotide sequence of SEQ ID NO: 6, or a polynucleotide complementary thereto; and
a primer or probe capable of amplifying or binding to a polynucleotide consisting of 10 to 100 consecutive nucleotides including a single nucleotide polymorphism (SNP) at nucleotide position 26 in the nucleotide sequence of SEQ ID NO: 8, or a polynucleotide complementary thereto.

18. The composition of claim 1, wherein the metabolic disease is obesity, type 2 and type 1 diabetes, hyperlipidemia, hypertension, non-alcoholic and alcoholic fatty liver disease, liver cancer, arteriosclerosis, or enteritis.

19. A kit for diagnosing metabolic disease susceptibility comprising the composition of claim 1.

20. A method of providing information for diagnosing metabolic disease susceptibility, comprising a step of detecting a gene or promoter variant, or a protein variant, a fragment thereof, or a polynucleotide encoding the same or measuring an expression level thereof in Akkermansia muciniphila in an isolated biological sample.


SEQUENCE LISTING (8 sequences)

SEQ ID NO: 1 - protein, 295 aa, synthetic construct
  REGION 1..295: note = Amino acid sequence of TEM type beta lactamase
MVCSCALSLGVMPVVAASPAADSAAVQKLAHSLKARVGMAAVMLDTGEAVAVGDETAYPMQSVFKFVLALSVLKRVDQGALNLEQIIHIRPEQLVKDTWSPLRERFPQGGDFSLKELLRVTVQESDNNTCDLLFALIGGTETVQKDLKEWGISGINVRFTEDEMLRNHELQYANSSRPSAMNALLRAFDEGKILNKDTQRLLWDMMAGCATGTTRLKGKLPQDYVVAHKTGSGFTTPEGVITAVNDVGIIVLPNGRKMAVSVFVMDSKDSAPACEKVIASMARWLCTEWQAAAAK

SEQ ID NO: 2 - DNA, 888 bp, synthetic construct
  misc_feature 1..888: note = polynucleotide sequence of TEM type beta lactamase
  source 1..888: mol_type = other DNA
atggtgtgctcctgcgctctttctctgggtgttatgcccgtggtggccgcgtctcccgcggctgattccgccgctgtgcagaagctggctcattccctgaaagcccgtgtgggcatggccgcagtgatgctggacacgggggaggccgtggcggtaggtgatgagactgcctatcccatgcagagcgtcttcaaattcgtgctggccctgtccgtgctcaagcgggtggaccagggagccctgaatctggaacagatcatccatatacgcccggagcagttggtaaaagacacgtggagtcccctgcgggagcgctttccgcagggcggtgatttttctttaaaggaactgctgcgcgtgaccgttcaggagagcgacaacaacacgtgcgacctcctgtttgccctcatcggcgggacggagaccgtgcagaaggatttgaaggaatggggcatcagcggcatcaacgtccggtttacggaagacgagatgctgcggaatcatgaattgcagtacgccaactcgagccgtccgtcagccatgaatgccctgctgcgcgcgtttgacgagggtaagatactgaacaaggacacgcagcggcttctctgggacatgatggccggctgcgctacggggactacgcgcctgaaagggaagttgccgcaggattatgtggtggcgcacaagaccggatcgggcttcaccactccggagggcgtgatcaccgccgtcaatgacgtcggcatcatcgttcttcccaatgggcggaaaatggcggtctccgtgttcgtcatggattcaaaggattccgctcccgcctgcgagaaggtgatcgcctccatggcccgctggctgtgcacggaatggcaggccgccgccgccaagtag

SEQ ID NO: 3 - protein, 639 aa, synthetic construct
  REGION 1..639: note = Amino acid sequence of PurF
MSDFLKHECGVAAIRLLKPLSYFQDKYGSTLWAFNKLLILMEKQYNRGQDGAGIGCVKLNMPLGQPYLFRTRDASKDALTTIFNGQIKKLNKKVRRGQVNLKDAEDIKNNFDYGGEILMGHLRYGTSGLFDEGSCHPYLRRTNWPTRTLMVLGNFNMTNTPELNQRMIERGQHPVFGTDTQTVLEEIGYHLDEAHTDLYRALRDSGMPGPEIPHAISSRLNIQEIIHNSARQWDGGYAIMGAIGNGDYFCLRDPHGIRPCHYLITDEFIAVASERVPLMTVFEVESEQVQELPPANMLSIKADGTHVITEFTTPLKPTPCSFEKIYFSRGNDPIVYRERKALGAALTPQIVDSLEDRFDKSAITYIPNTAETAYYGLLEGLRVYRRKRVHAQLLEALRNGTLDENMLDSAILKRWPRGEKIAHKDIKMRTFITQEKSRAQLVSHVYDLTYGAVGPEDVLVAIDDSIVRGTTLRRSILRILGRTNPRKIVVASTAPQIRYPDCYGIDMSELGNFIAFQAAVSLIKQHGQARLLEEVYKACKEELTKPREERRNCVKAIYEGLTDAEISREITRLVTPHDAPCPVEVIFQTIENLHESIEGPCGDWYFTGDYPTPGGYTTVNVAYMRWFEGKGGRAYDLPL

SEQ ID NO: 4 - DNA, 1920 bp, synthetic construct
  misc_feature 1..1920: note = polynucleotide sequence of PurF
  source 1..1920: mol_type = other DNA
atgagcgattttcttaaacacgagtgcggcgtagccgccattcgtctgcttaagcctctctcttattttcaggataaatacggctccaccctctgggcgttcaacaagctgctcatcctgatggaaaagcagtacaaccgcggccaggatggcgcgggcatcggctgcgtgaaactgaacatgcccctcggccagccctacctgttccgcacccgggacgccagcaaggacgccctcaccaccatcttcaacgggcaaatcaagaagcttaacaaaaaagtccgccgcggtcaggtcaacctgaaagacgctgaagacattaaaaacaactttgactacggcggggaaatcctgatggggcacctccgctacggcacctccggcctctttgacgaaggctcctgccatccctacctgcgccgcaccaactggccgacgcgcaccctgatggtgctgggcaacttcaacatgaccaacacgccggaactgaaccagcgcatgattgagcgcggccagcatcccgtcttcggcacggacacgcagaccgtccttgaagaaatcggctaccatctggatgaagcccatacggacctttaccgcgccctgcgggacagcggcatgcccggcccggaaatcccccatgccatctcctcccggctcaacatccaggaaatcattcacaactcagccaggcagtgggacggcggctacgccatcatgggcgccatcggcaacggggactacttctgcctgcgggacccgcacggcatccgcccctgccactacctgattacggatgaattcattgccgtggcctctgaacgcgtcccgctgatgaccgtctttgaagtggaaagcgaacaggtgcaggaactgccgccggccaacatgctttccatcaaggcggacggcacgcacgtcatcacggaattcaccacgcccctgaaacccacgccgtgttcctttgaaaagatatacttctcccgcgggaacgaccccatcgtgtaccgggaacgcaaggccctgggcgcggcgctgaccccgcagatcgtggactccctggaagaccgctttgacaaatctgccatcacctacattcccaacacggcggaaaccgcctactacggcctgctggaaggcctgcgcgtctaccgccgcaagcgcgtgcacgcccagctactggaagccctgagaaacgggaccctggatgaaaacatgctggacagcgccatcctgaaacggtggccgcgcggtgaaaaaatcgcccacaaggacatcaagatgcgcaccttcatcacgcaggaaaagagccgcgcgcagcttgtctcccacgtatatgacctcacctacggggccgtgggaccggaagacgtgctcgtcgccattgacgactccatcgtccgcggcaccacgctgagaaggtccatcctccgcatcctgggccgcaccaatccgcggaaaatcgtcgtcgcctccaccgctccgcaaatccggtatccggactgctacggcattgacatgtccgaactggggaacttcatcgccttccaggcggcagtctccctgatcaagcagcacgggcaggcccggttgctggaagaagtatacaaggcatgcaaggaggaactgaccaagcccagggaagaacggcgcaactgcgtcaaggccatttacgaaggcttgacggacgctgaaatctcccgtgaaatcacccgcctggtcactccgcacgacgccccgtgccccgtggaagtcatcttccagaccatcgaaaacctgcacgaatccatagaaggcccctgcggcgactggtatttcaccggagactacccgactccgggcggatataccacagttaatgtggcatacatgcgctggtttgagggtaaaggggggcgtgcatacgatttgcccttgtag

SEQ ID NO: 5 - protein, 235 aa, synthetic construct
  REGION 1..235: note = Amino acid sequence of PurQ
MPHALLLKYPGTNCDAETERALRAAGFSTQIQPIATATPEHVAKSQLVVLGGGFSYGDYVTSGRLAQLETERFLGDALHRHHAGGGLLLGICNGFQILTKLGILPDSSLIDNKKERFECVWAKLVKVAKNSPFLQGLPDEFELPSAHAEGRLVTRPGDAEKYLAAGNVALQYVDNRDGCECSIAGLQDSTGRAMGLMPHPERFLLPRHHYDKDWAGNEDWGWGYFLFKSAFDALK

SEQ ID NO: 6 - DNA, 708 bp, synthetic construct
  misc_feature 1..708: note = polynucleotide sequence of PurQ
  source 1..708: mol_type = other DNA
atgcctcacgcacttttactgaaataccccggcaccaactgtgacgcggaaacggaacgcgccctgcgcgcggccggcttctccacccagatccagcccatcgccacggccacgccggaacacgtcgccaaatcccagctcgtcgtcctcggcggcggcttcagctacggggactacgtcaccagcggccgcctggcccagctggaaacggaacgcttcctgggagacgccctgcaccggcaccatgccggaggcggcctgctcctgggcatctgcaacggcttccagatactcaccaagctggggattctgccggattccagcctgattgacaacaagaaggaacgttttgaatgcgtctgggccaagctggtgaaggtagctaaaaactcacccttcctccagggccttccggatgaattcgagctgccctccgcccacgcggaaggccgcctggtcacccgccccggagacgcggaaaaatacctggctgccggaaatgtggctctccagtacgtggacaaccgcgacggctgtgaatgcagcatcgcgggcctccaggattccaccggccgcgccatgggcctgatgccccacccggaacgcttcctgctgccccgccaccactatgacaaggactgggccggcaatgaagactggggctggggatacttcctcttcaaatccgcctttgacgccctgaaataa

SEQ ID NO: 7 - protein, 333 aa, synthetic construct
  REGION 1..333: note = Amino acid sequence of PurM
MSGKLTYKQSGVDTKEAAAFVSDISSHVKRTQKQRSLHQAFGLFAAAYDLSSYKEPVIVTGCDGVGTKTEILFELDMVETAGKDLVAMNVNDILTTGGDPLLFLDYLGISNLEQERTRITRLVAGMCDYLESCNCILAGGETAEMPGVVPESIVELSGFCIGCCEKSKLIDPKTVRPGDVFIGYKSDSFHANGWSLIRRILEENPDVVDEEELRSLLQPTRLYHDVVADMRRFNVIPKAYAHITGGGLPENLERFLGDYGADLTIPCWDNTAAQKILKHVDPQDRFNTFNMGIGWVAIVKPEDVEAALKAGPGGTVIGTLREGRGIHVKVQGE

SEQ ID NO: 8 - DNA, 1002 bp, synthetic construct
  misc_feature 1..1002: note = polynucleotide sequence of PurM
  source 1..1002: mol_type = other DNA
atgtccggcaaactaacctacaagcaatcgggggtcgataccaaagaagcagccgctttcgtatccgacatcagctctcacgttaaaagaacgcaaaagcagagatccctgcaccaggccttcggtctttttgccgccgcctatgacctgagctcctacaaggaaccggtcatcgtcaccggatgcgacggcgtaggcaccaagacggaaatccttttcgaactggacatggtggaaaccgccggcaaggacctggtggccatgaacgtcaacgacatccttaccacgggcggagaccctcttcttttcctggactacctgggcatctccaatctggaacaggaacgtacccgcatcacccgcctggtggccggcatgtgcgactatctggaatcctgcaactgcatcctggccggcggagaaacggcggaaatgcccggcgtggttccggaatccatcgtggaactctccggcttctgcatcggatgctgtgaaaaaagcaagctgatcgaccccaagaccgttcgccccggagacgtattcatcggctacaaatccgacagcttccatgccaacggctggagccttatccgccgcattctggaggaaaacccggacgtggttgatgaagaggaactccgttccctgctacagcccacccgcctgtaccatgacgtggtggcggacatgcgccgcttcaacgtcatcccgaaggcatacgcccacatcacggggggcggccttccggaaaacctggaacgcttcctgggcgactacggcgcggacctcaccattccctgctgggacaacaccgcagcccagaaaatcctgaagcatgtggacccgcaggaccgcttcaacacctttaacatgggcatcggctgggtagccatcgtgaagccggaggacgtggaagccgcgctgaaggcaggccccggcggcaccgtcatcggcacgttgagagaaggccgcggcattcatgtcaaggtacagggcgaatag